(19) United States
(12) Patent Application Publication
(10) Pub. No.: US 2023/0034731 A1
(43) Pub. Date: Feb. 2, 2023

(54) SPINAL IMPLANTS FOR MESH NETWORKS

(71) Applicant: Carlsmed, Inc., Carlsbad, CA (US)

(72) Inventors: Michael J. Cordonnier, Carlsbad, CA (US); Niall Patrick Casey, Carlsbad, CA (US); Shariq Hussain, Vista, CA (US)

(21) Appl. No.: 17/856,625

(22) Filed: Jul. 1, 2022

Related U.S. Application Data
(60) Provisional application No. 63/218,190, filed on Jul. 2, 2021.

(51) Int. Cl. G16H 50/20; A61B 5/00

(52) U.S. Cl. CPC G16H 50/20; A61B 5/4566

(57) ABSTRACT

Systems and methods for establishing and managing a patient-device network on or about a body of a patient are disclosed. One or more of the devices implanted into, affixed to, and/or carried by a patient may be configured to establish and manage a communication network. For example, one or more of the implants may include networking mechanisms that autonomously connect to each other, thereby establishing and managing a mesh network or an ad-hoc network on or about a portion of the patient body. The networked devices can communicate with each other and/or to other external devices.

### CROSS-REFERENCE TO RELATED APPLICATION

The present application claims priority to U.S. Provisional Patent Application No. 63/218,190, filed Jul. 2, 2021, the disclosure of which is incorporated by reference herein in its entirety.

### TECHNICAL FIELD

The present disclosure is generally related to communicatively coupling medical devices, and more particularly to systems and methods for mesh networks including implanted medical devices, including intervertebral implants.

### BACKGROUND

Orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis. Unfortunately, conventional orthopedic implants, such as intervertebral discs and fixation rods, do not actively work together to provide post-operative real-time adjustments and corrections.

In addition, numerous types of data associated with patient treatments and surgical interventions are available. To determine treatment protocols for a patient, physicians often rely on a subset of patient data available via the patient's medical record and historical outcome data. However, the amount of patient data and historical data may be limited, and the available data may not be correlated or relevant to the particular patient to be treated. Additionally, although digital data collection and processing power have improved, the collection mechanisms tend to be limited to one physiological trait and/or one disease/condition. For example, conventional technologies in the field of orthopedics may be limited to a limited set of devices and unable to utilize other patient data or pre-treatment data. Additionally, such data may not be used by conventional implanted devices, which may also be unable to communicate with each other to coordinate their operation based on current disease state/condition. Thus, conventional technologies are limited in collecting data and generating and optimizing patient-specific treatments (e.g., surgical interventions and/or implant designs) to achieve favorable treatment outcomes.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a computing system for establishing/managing a patient-device network, according to an embodiment of the present technology.

FIG. 2 illustrates a patient device suitable for use in connection with the system of FIG. 1, according to an embodiment of the present technology.

FIG. 3A is a flow diagram illustrating a method for providing patient-specific medical care, according to an embodiment of the present technology.

FIG. 3B illustrates a mesh network system including implanted patient devices, according to an embodiment of the present technology.

FIGS. 4A-4C illustrate exemplary data sets that may be used and/or generated in connection with the methods described herein, according to an embodiment. FIG. 4A illustrates a patient data set. FIG. 4B illustrates reference patient data sets. FIG. 4C illustrates similarity scores and outcome scores for the reference patient data sets of FIG. 4B.

FIG. 5 is a flow diagram illustrating another method for providing patient-specific medical care, according to an embodiment.

FIG. 6 is a partially schematic illustration of an operative setup and associated computing systems for providing patient-specific medical care, according to an embodiment.

FIGS. 7A-7D illustrate an exemplary patient data set that may be used and/or generated in connection with the methods described herein, according to an embodiment.

FIGS. 8A and 8B illustrate an exemplary virtual model of a patient's spine that may be used and/or generated in connection with the methods described herein, according to an embodiment.

FIGS. 9A-1-9B-2 illustrate an exemplary virtual model of a patient's spine in a pre-operative anatomical configuration and a corrected anatomical configuration. More specifically, FIGS. 9A-1 and 9A-2 illustrate the pre-operative anatomical configuration of the patient; and FIGS. 9B-1 and 9B-2 illustrate the corrected anatomical configuration.

FIG. 10 illustrates an exemplary surgical plan for a patient-specific surgical procedure that may be used and/or generated in connection with the methods described herein, according to an embodiment.

FIG. 11 illustrates an exemplary surgical plan report detailing the surgical plan shown in FIG. 10 for surgeon review and that may be used and/or generated in connection with the methods described herein, according to an embodiment.

FIGS. 12A and 12B illustrate an exemplary patient-specific implant that can be used and/or generated in connection with the methods described herein, according to an embodiment.

FIG. 13 illustrates a segment of a patient's spine after several patient-specific implants have been implanted therein, according to an embodiment.

FIG. 14 shows a patient's spine and a remote device for controlling actuation of intervertebral implants, according to an embodiment.

FIG. 15 illustrates an exemplary corrective plan that may be used and/or generated in connection with the systems and methods described herein, according to an embodiment.

FIGS. 16A-16D show a patient's spine in different configurations, according to an embodiment.

### DETAILED DESCRIPTION

The present technology is directed to systems and methods for establishing and managing a network among patient-devices. One or more of the devices implanted into, affixed to, and/or carried by a patient may be configured to establish and manage a communication network. For example, one or more of the implants may include networking mechanisms that autonomously connect to each other, thereby establishing and managing a mesh network or an ad-hoc network on or about a portion of the patient body. The networked devices can communicate with each other and/or to other external devices.

Advances in modern technology and medicine has allowed for various devices to be implanted into, attached on, or carried by the human body. Some devices, such as implants, may be located at a fixed location on a patient permanently or for a relatively long duration of time (e.g., years, decades, etc.). Some examples of affixed devices or implants may include spinal implants (e.g., artificial discs, interspinous spacers, intervertebral cages, etc.), orthopedic implants (e.g., artificial hips, fracture repair structures, alignment inserts, spinal-assistance structures, corresponding attachment mechanisms, etc.), sensory/neurological implants, replacement organs, assistive mechanisms (e.g., pacemakers, defibrillators, valves, stents), or the like. Other devices, such as attachable or wearable devices (e.g., blood glucose monitors, heart monitors, etc.), may be attached to or worn on the patient body for significant durations. Still other devices (e.g., personal devices, such as mobile phones, smart watches, and/or other personal health monitors) may be carried by the patient or within a fixed distance from the patient for significant portions of each day.

However, conventional devices are designed and/or associated with a user for specific physiological traits and/or specific diseases/conditions. Accordingly, the conventional devices are unable to connect to other devices without additional or invasive procedures, such as surgery. Moreover, even when devices are configured to communicate with other devices, the connectible devices are limited to certain types of devices and/or concurrently implanted/attached devices.

Several embodiments of the present technology may include a networking mechanism configured to autonomously establish and manage a network among patient-devices (e.g., wearable devices, attached devices, and/or implants). The networking mechanism can include a wireless communication circuit, a control circuit, a power management circuit, and/or a storage circuit. In some embodiments, the networking mechanism can include radio-frequency identification (RFID) mechanisms that can wirelessly communicate with other devices and/or derive operating power from the wireless signals. Otherwise, the power management circuit can be configured to communicate with, discover, and/or connect to nearby devices according to a stimulus (e.g., a trigger signal from an external device, a detected physiological state of the patient) and/or a predetermined frequency. The communicating devices (e.g., the networked devices) can be limited by a communication range that corresponds to a frequency and/or a signal strength preset for the communication protocol. Upon each communication, the devices can store and/or update networking details, such as by adding newly discovered device identifiers, removing undetected device identifiers, and/or other communication details. Accordingly, the networking mechanism can establish and maintain a mesh network and/or an ad-hoc network of patient personal devices.

The patient devices can include sensors and/or circuits configured to determine conditions of the devices and/or physiological conditions of the corresponding patient. Some examples of the sensors may include mechanical sensors that can sense (e.g., detect, measure, etc.) forces, moments (e.g., bending moments), loads, contact, distance(s), pressures (e.g., contact pressure, fluid pressure, etc.), height, orientation/position (e.g., in tracking lordosis, spine curvature, etc.), or the like. Other example sensors may include temperature sensors, biochemical sensors, optical sensors, chemical, or the like.

The patient devices can use the networking mechanism to communicate and provide the sensor readings and/or the device statuses to other networked devices and/or connected external devices. Some example adjustments can include mechanical adjustment corresponding to local adjustments (e.g., single device adjustment), regional adjustments (via, e.g., a reduced set of devices), and/or global adjustments (via, e.g., a larger set or a full set of devices). Other example adjustments can include biochemical adjustments (by, e.g., injecting or removing biochemical agents), such as for promoting bone growth, healing/recovery, disease prevention, treatment, and/or accurate adjustments to account for patient changes (e.g., disease progression and/or improvements).

In some embodiments, the communicating set of devices can be configured (via, e.g., one master device and/or a distributed set of processes) to analyze the sensor outputs and/or device statuses of the networked devices. For example, a set of networked implants (e.g., spine orthopedic implants) can analyze whether one or more of the implants have successfully fused or established targeted conditions. Also, a set of networked devices can track a progression of one or more diseases/conditions.

Additionally or alternatively, the patient devices can include actuators and/or adjustment mechanisms that can effect changes on the device configuration/structure and/or on the body of the user. For example, orthopedic implants may be configured to adjust structural conditions and/or apply force(s) to the body of the user. The patient devices can be adjusted according to control signals that result from the analysis. Continuing with the example, the orthopedic implants can receive control signals via the networking mechanism. In response, the orthopedic implants can implement post-operative adjustments. Similarly, other patient devices can adjust operations and/or configurations according to the current physiological state of the patient and/or the progression of a targeted disease/condition.

In some embodiments, patient devices can include one or more electrodes configured for neuromodulation, energy therapy (e.g., radiofrequency), generating fields (e.g., magnetic fields), or the like. For example, neuromodulation patient devices can include one or more pulse generators, controllers, leads, power source(s), charging elements, or the like. The neuromodulation patient devices can be programmed to provide monopolar modulation, bipolar modulation, tripolar modulation, or other suitable modulation to, for example, create the sensation of paresthesia, which can be characterized as an alternative sensation that replaces pain signals sensed by the patient. In some embodiments, a neuromodulation patient device is in the form of an intervertebral cage or artificial disc with electrodes that can be implanted adjacent to a nerve tissue (e.g., spinal cord, ganglion nerve roots, etc.) or accessible nerve tissue near or adjacent to the spine. In some embodiments, the patient devices include electrodes configured to emit RF energy to damage, ablate, or otherwise disrupt tissue. A patient device can include a single electrode for monopolar RF energy delivery or plurality of electrodes for biopolar RF energy delivery. In some embodiments, patient devices have electrodes that work together to provide biopolar RF energy or other types of energy to tissue between the devices in combination with mechanical adjustments and/or bio biochemical adjustments

In some embodiments, an implanted networked system can operate to, for example, provide predetermined height restoration, lordosis angle correction, and/or coronal angle correction. The implanted networked system can include patient devices that communicate with another via a wired connection, wireless connection, or the like. The implanted networked system can store programs, settings, protocols, data, plans (e.g., treatment plans, correction plans, etc.), schedules, etc. The programs can include calibration programs, networking programs, encryption/decryption programs, or the like. The settings can include configuration settings, position settings (e.g., implant settings, post-operative settings, etc.), or the like. The protocols can include networking protocols, communication protocols, new device detection protocols, token management protocols, or the like. The data can include collected data (e.g., data from sensors, data retrieved from external devices, etc.), treatment plan data, tracking data (e.g., lot number, batch number, etc.), device configuration data, surgical operation data, or the like. In some implementations, each implantable device includes a networking mechanism for inter-device communications for implementing a treatment plan over a period of time, correction plan, etc. The correction plan can include target anatomical positions or configurations suitable for the patient. The correction plan can be modified based on disease progression, changes in patient health, patient improvement, etc.

In some embodiments, a patient device has a networking mechanism that can implement a predetermined communication protocol to establish and manage the device network. For example, the networking mechanism can use a predetermined frequency, bandwidth, data rate, symbol set, encoding/decoding, or the like for the communicated signals. Also, the networking mechanism can use a predetermined data format, such as for reference/pilot signal, information mapping or sequence, or the like. Further the networking mechanism can utilize predetermined messaging sequences and corresponding timings, multiplexing or collision avoidance mechanisms, or the like to establish and manage the network. For example, the networking mechanism can include requirements or sequences for discovery, authentication, network configuration, power management, messaging details, or the like. In some embodiments, the networking mechanism can utilize one or more established communication protocols, such as Bluetooth, WiFi, Near-Field communication (NFC), ZigBee, Z-Wave, etc. In other embodiments, the networking mechanism can use or define a communication protocol configured specifically for implants and/or other patient devices.

In some embodiments, the networking mechanism can establish a communicating order, frequency, and/or time slot for each of the networked devices. For example, the networking mechanism can establish a communication controller (e.g., a master device) that controls and/or enables each device for communication. One or more of the devices may function as the communication controller. The communication controller may function as a conduit that facilitates communication between slave devices and/or with external devices. In other embodiments, the networking mechanism can implement a distributed control mechanism, such as according to a registration sequence for the networked devices and/or according to a collision-avoidance protocol. For example, the networked devices may broadcast messages that each include an identifier of the recipient device. All of the devices can monitor the transmitted message and the intended recipient device can store and process the broadcasted message. When two or more devices simultaneously transmit (e.g., a collision event), the colliding devices can detect the collision based on the receiving/listening portion of the device. When collisions are detected, the colliding devices can each be configured to wait a random duration (selected via, e.g., a random number generator local to the device) and retransmit the message.

One or more of the networked devices may be configured to communicate with external devices. The externally-interfacing device(s) may be configured to provide measurements and/or states of the networked devices to the external devices. Also, the externally-interfacing device(s) may be configured to receive control signals from the external devices.

As an illustrative example, the networking mechanism may be implemented across a set of orthopedic implants used to adjust or correct the spine of a patient. Each of the implants can include a set of sensors, a communication circuit, a processor, a memory, and/or an actuator. The implants can establish the mesh network and communicate with each other as described herein. Each of the implants can determine any structural changes, relative locations/distances between devices (via, e.g., reference signal strengths), orientation/pose, or the like. The determined information can be communicated between the networked devices and/or with external processing device. Accordingly, the determined information may be processed to detect any changes in the spine of the patient. Also, one or more of the implants may be used to affect the spine (via, e.g., the actuators, neuromodulation, expansion/contraction, and/or biochemical agents) to implement post-operative adjustments, to promote healing or corrections for the patient, etc.

In some embodiments, at least one of the orthopedic implants is a patient-specific implant designed according to a surgical treatment plan. The patient-specific implant can store the surgical treatment plan or a portion thereof. The surgical treatment plan can include native anatomy data (e.g., images, scans, etc.), corrected or target anatomy data, patient history, identification data (e.g., patient identification data, device data, etc.), data received from other devices, physician notes, or the like. The network implants can cross communicate to maintain a proper anatomical state or condition. In some embodiments, a treatment protocol can be performed. For example, if a patient experiences discomfort due to nerve tissue compression, and implant in the form of an artificial disc can expand to increase distancing between vertebrae, thereby reducing nearby nerve compression. In some implementations, if the artificial disc detects improper movement that typically causes nerve compression, a neuromodulation protocol can be performed to limit, minimize, or prevent discomfort. This allows the network implants to cooperate to achieve one or more treatment goals.

Embodiments of the present technology will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

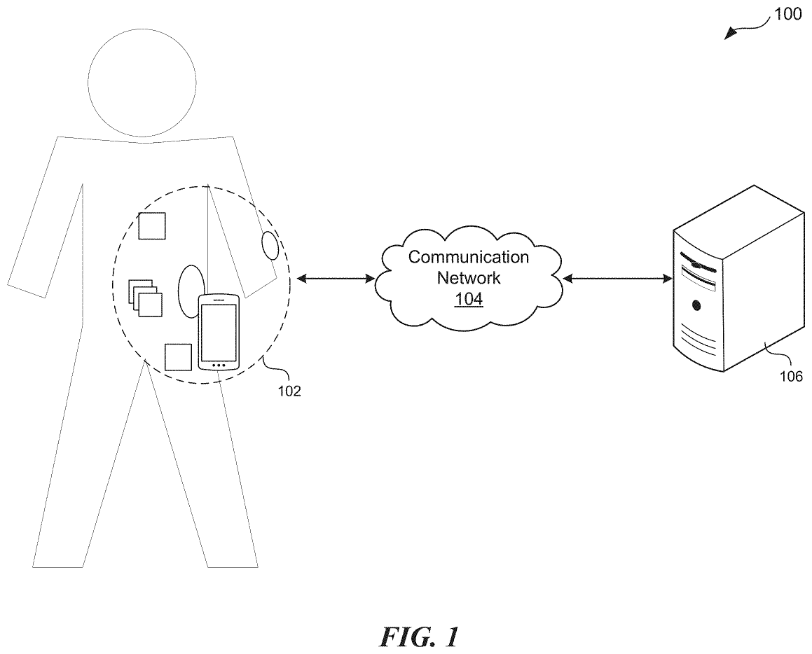

FIG. 1 is a network connection diagram illustrating a computing system 100 for establishing/managing a patient-device network 102 according to an embodiment of the present technology. The patient-device network 102 can be for communicatively coupling devices (e.g., implants, wearables, and/or other personal or health-monitoring devices) attached to or carried on the body of a patient. Accordingly, the patient-device network 102 can be located on and/or be defined relative to the body of the patient. Some examples of the coupled devices can include orthopedic implants, joint/organ implants, auxiliary implants (e.g., pacemakers, stints, etc.), heart monitors, continuous blood glucose monitors, smart watches, smart phones, wearable health monitors, or the like. The patient-devices in the network 102 may include one or more processors, and memory storing instructions executable by the one or more processors to perform the methods described herein, such as to establish and manage the patient-device network 102.

The patient-device network 102 can be coupled to an external device 106 (e.g., a computing device external to the patient-device network 102) through a communication network 104. Some examples of the external device 106 can include workstations, personal computers, and/or servers. The external device 106 can be associated with a healthcare provider that is treating the patient. Although FIG. 1 illustrates a single external device 106, in alternative embodiments, the external device 106 can instead be implemented as a computing system encompassing a plurality of computing devices. In some embodiments, the external device 106 can correspond to a device used to perform medical analysis and/or patient-device control.

The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

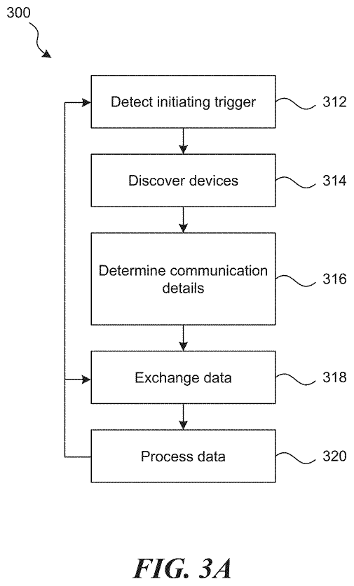

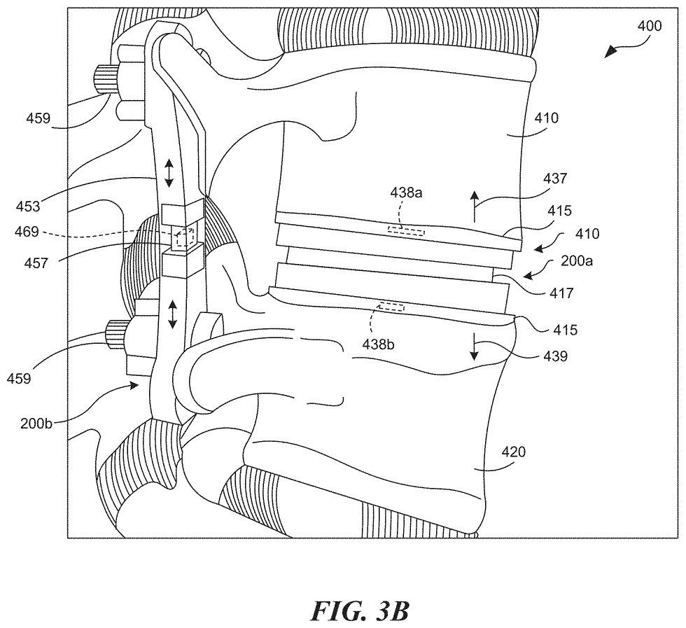

It shall be appreciated that the components of the system 100 can be configured in many different ways. For example, in alternative embodiments, the personal or mobile computing devices (e.g., smart phones and/or personal computers) may connect to the patient-device network 102 and facilitate a connection to the communication network 104. Also, the personal device may function as a communication master and/or trigger one or more operations of the networked devices. Systems 100 can include the devices, technology, and features are discussed in connection with FIG. 3B.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

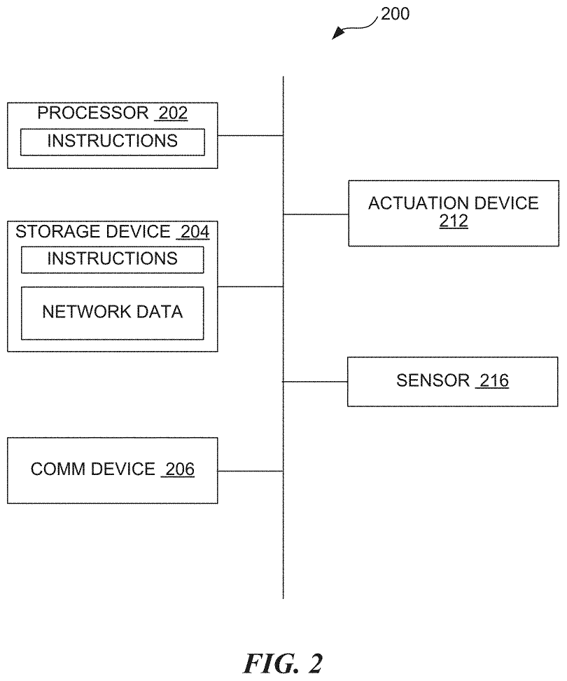

FIG. 2 illustrates a patient device 200 (e.g., an implant, a wearable device, a health-monitoring device, a personal device, etc.) suitable for use in connection with the system 100 of FIG. 1, according to an embodiment of the present technology.

In some embodiments, for example, the patient device 200 can include electronic/electrical devices, such as one or more processors 202, one or more storage devices 204, one or more communication devices 206, one or more actuation devices 212, one or more sensors 216, or a combination thereof. The various devices can be coupled to each other via wire connections and/or wireless connections. For example, the patient device 200 can include a bus, such as a system bus, a Peripheral Component Interconnect (PCI) bus or PCI-Express bus, a HyperTransport or industry standard architecture (ISA) bus, a small computer system interface (SCSI) bus, a universal serial bus (USB), an IIC (I2C) bus, or an Institute of Electrical and Electronics Engineers (IEEE) standard 1394 bus (also referred to as “Firewire”). Also, for example, the patient device 200 can include bridges, adapters, processors, or other signal-related devices for providing the wire connections between the devices. The wireless connections can be based on, for example, cellular communication protocols (e.g., 3G, 4G, LTE, 5G, etc.), wireless local area network (LAN) protocols (e.g., wireless fidelity (WIFI)), peer-to-peer or device-to-device communication protocols (e.g., Bluetooth, NFC, RFID, etc.), Internet of Things (IoT) protocols (e.g., NB-IoT, LTE-M, etc.), and/or other wireless communication protocols.

The processors 202 can include data processors (e.g., central processing units (CPUs), special-purpose computers, and/or onboard servers) configured to execute instructions (e.g., software instructions) stored on the storage devices 204 (e.g., computer memory). The processors 202 can implement the program instructions to control/interface with other devices, thereby causing the patient device 200 to execute actions, tasks, and/or operations.

The storage devices 204 can include non-transitory computer-readable mediums having stored thereon program instructions (e.g., software). Some examples of the storage devices 204 can include volatile memory (e.g., cache and/or random-access memory (RAM)) and/or non-volatile memory (e.g., flash memory and/or magnetic disk drives). Other examples of the storage devices 204 can include portable memory drives and/or cloud storage devices.

In some embodiments, the storage devices 204 can be used to further store and provide access to processing results and/or predetermined data/thresholds. For example, the storage devices 204 can store networking data, such as identifiers of other devices in the network, communication detail (e.g., assigned frequency, ordinal/time slot, etc.), or the like. The storage devices 204 can be within sealed housings or components to prevent contamination or contact with body fluids.

The communication devices 206 can include circuits configured to communicate with other devices (e.g., devices having separate physical housing/casing). For example, the communication devices 206 can include receivers, transmitters, modulators/demodulators (modems), signal detectors, signal encoders/decoders, antennas, etc. The communication devices 206 can be configured to send, receive, and/or process electrical signals according to one or more communication protocols as described above. In some embodiments, the patient device 200 can use the communication devices 206 to exchange information between other devices in, on, or carried by the body of the user. Accordingly, the communication devices 206 can facilitate the establishment and management of the patient-device network 102. Also, the patient device 200 can use the communication device 206 to connect to the communication network 104 of FIG. 1, thereby facilitating communication with the external device 106 of FIG. 1.

The patient device 200 can include the actuation devices 212 (e.g., motors, actuators, wires, artificial muscles, electroactive polymers, etc.) configured to drive or manipulate (e.g., displace and/or reorient) one or more structural aspects of the patient device 200. For example, the actuation devices 212 may be configured to apply force on the body of the patient and/or release or remove a biochemical agent within the patient as described above.

The sensors 216 can be configured to obtain information about the patient device 200 and/or the surrounding environment thereof. For example, the sensors 216 can include load/force sensors, pressure sensors, proximity sensors, location sensors, gyroscopes, accelerometers, biochemical sensors, self-test circuitry, or the like.

FIG. 3A is a flow diagram illustrating a method 300 for establishing and managing a network (e.g., the patient-device network 102). The method 300 can be for implementing the networking mechanism using the patient devices.

At block 312, the computing system 100 of FIG. 1 (via, e.g., one or more of the patient devices) can detect an initiating trigger to establish and/or manage the patient-device network 102. For example, the patient devices can include an internal timer for initiating the trigger. Also, the patient devices can detect the trigger when wireless signal provides sufficient power to initialize circuits (e.g., NFC devices) and/or includes an initialization command.

At block 314, the computing system 100 can discover devices for networking purposes. The computing system 100 can use a predetermined protocol for discovery. For example, in response to the trigger, one or more of the devices can generate a random number (e.g., bounded by a minimum and a maximum) for a wait duration. After waiting the randomly generated length of time, the device(s) can transmit an initial message that includes the device identifier of the sender. The remaining devices can receive the transmit message and recover the device identifier. The listening devices can redetermine the random duration and/or use the previously generated duration to transmit the message. The process can repeat until all of the devices have transmitted a message. The discovered devices may conclude the discovery process when no transmissions are detected for a predetermined duration (e.g., a duration greater than the maximum boundary of the random duration).

In some embodiments, one or more of the devices (e.g., the device transmitting the trigger/power signal and/or the first discovered device) can be configured to function as a communication host or master. For such embodiments, the host device can transmit a message that notifies or commands the end of the discovery process.

When two or more devices simultaneously transmit, each of the transmitting devices can listen to the transmit message to detect a collision, such as when received message does not match the transmitted message. If collision is detected, each of the transmitting devices can redetermine the random length and retransmit the message after waiting the random length.

Once the patient-device network 102 is initialized, the patient devices can use the previous registration/communication order as a starting point (e.g., without using the randomly generated wait duration) for registration or validation. Accordingly, the devices can sequentially transmit before the minimum required duration for the random wait. Thus, any devices newly introduced into the environment can transmit at the end of the pre-existing sequence, and the networking mechanism can add the new devices to the end of the sequence.

At block 316, the computing system 100 can determine communication details, such as for determining multiplexing mechanisms. For example, the discovered patient-devices can store the identifiers of other discovered devices and/or the sequence in which the devices transmitted the discovery messages. The patient-devices can may use the sequence to designate a resource (e.g., a time slot and/or a frequency) for transmitting messages. In other words, the first-discovered device can transmit using a first slot/frequency, the second-discovered device can transmit using the second slot/frequency, and so on.

At block 318, the computing system 100 can exchange (e.g., transmit and/or receive) data at one or more of the networked devices. The networked devices can transmit messages according to the communication details. The transmitted messages can be broadcasted to all devices in the patient-device network and/or the external device. The transmitted message can include a recipient identifier. The remaining devices can analyze the recipient identifier and receive the remainder of the message accordingly. In some embodiments, the receiving device can reply with an acknowledgement message to complete the exchange. The patient-devices can thus exchange any sensor readings, device statuses, commands (e.g., reconfiguration or actuation commands), network management messages, and/or other message content within the patient-device network 102 and/or with the external device 106.

At block 320, the computing system 100 can process the communicated data. For example, the device receiving the transmitted messages (e.g., a designated analysis device in the patient-device network 102 and/or the external device 106) can analyze the sensor outputs according to a predetermined mechanism. Accordingly, the computing system 100 can determine a targeted information (e.g., patient recovery, progression of disease, etc.) regarding the patient and/or the devices. In some embodiments, the analyzing device can further derive corrective actions according to the determined current condition. The corresponding commands/settings for the corrective actions can be sent/exchanged during a subsequent round of communication.

FIG. 3B illustrates a mesh network system 400 (“system 400”) including implanted patient devices 200a and 200b (collectively “patient device 200”) in accordance with some embodiments of the present technology. The implanted patient devices 200 can wirelessly communicate with one another for coordinated operation to provide one or more spinal corrections. New devices or external devices can communicate with the patient devices 200 using tokens, key-based authentication, or another authentication protocol. This allows new devices to be added to the system 400 suitable for use in connection with the system 100 of FIG. 1 and method of FIG. 3A, as well as other embodiments disclosed herein. The description of the patient devices 200 of FIG. 2 applies equally to the patient devices 200 of FIG. 3B unless indicated otherwise.

The system 400 can be programmed to prevent setting changes, data manipulation, or the like from any device that is not part of the communication network and can provide locked communication channels, encrypted communications, combinations thereof, or the like. The networked implant system 400 can store data, including plans (e.g., surgical plans, treatment plans, disease progression plans, etc.), diagnostic protocols, kinematic relationships, mathematic rules, lookup tables, association of conditions/disease to plans, etc. for coordinating operation. The networked implant system 400 can include, without limitation, static implants (e.g., rods, cages, fixed configuration spacers, etc.), dynamic implants (e.g., artificial discs, articulating joints, etc.), non-expanding devices, and/or expanding devices (e.g., internally or externally powered devices, pneumatic devices, self-expanding devices, etc.). The number, configuration, positions, and operability of the devices can be selected based on, for example, treatment plan, targeted correction, predicted disease progression, combinations thereof, or the like.

The networked implant system 400 can be configured to implement corrective treatment plan(s) and/or to correct for deformities of the spine described by, without limitation, lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.), pelvic parameters, combinations thereof, or the like. The networked implant system 400 can store, for example, algorithms that use collected data (e.g., sensor readings, device setting/configuration data, etc.) as inputs used to determine the pathology (e.g., discs height, segment flexibility, bone quality, rotational displacement), and the algorithm can use these additional inputs to further define optimal implant configurations for the current pathology. To treat multiple disease/conditions, the networked implant system 400 can predict outcomes for one or more conditions/diseases based on adjustments. The predicted outcomes can then be ranked, categorized, weighted, aggregated, etc. to evaluate the patient's condition/state and determine whether to adjust implant configurations, adjustment protocols, patient monitoring plans, etc. This allows conditions/diseases to be ranked and prioritized. In some embodiments, the implants can collect data to track fatigue or service life. In some embodiments, the networked implant system 400 uses sensor readings for diagnostics.

The networked implant system 400 can automatically or at least semi-automatically determine a corrected anatomical configuration for the patient based on data, including, for example, collected data, implant data (e.g., current configuration, implant location, current position, etc.), patient data (e.g., current age, surgical plan data, etc.), and/or patient data. The data can be inputted into one or more algorithms or rules for determining parameters or values. Such data can include anatomical values (e.g., lumbar lordosis, Cobb angles, etc.), comparison data, historical data, or lookup tables (e.g., prior patient data), etc. The networked implant system 400 can determine anatomical corrections for the patient that can be achieved based on capabilities (e.g., available expansion, contraction, articulation, etc.) of the implanted devices. When additional devices are implanted or join the network 400, programming, plans, schedules, or treatments can be updated, thereby incorporating the additional devices into the overall treatment plan.

The patient devices 200 can be expanded or contracted over a period of time to treat a patient suffering from, for example, one or more deformities. The patient devices 200 can be reconfigured based on a predetermined schedule, triggers, or patient condition changes, etc. In some implementations, the implants can be reconfigured when a predetermined state or condition is achieved. This allows for mechanical corrections when the patient's disease or condition improves (e.g., improvements due to rehabilitation, strengthening body, etc.). In some implementations, one or more of the implanted devices reconfigure themselves according to a treatment schedule to gradually move anatomical elements to target positions.

The patient devices 200 can be moved concurrently or sequentially to provide targeted corrections. Additional sensor data can be collected during this process to monitor adjustments. If applied loads reach maximum allowable values, the patient devices 200 can reduce actuation speeds or determine an alternative target configuration. The networked implant system 400 then determines configurations for one or more of the patient devices 200 to provide the target anatomical configuration or correction. In some implementations, the networked implant system 400 can generate the target anatomical configuration or correction based on sensor readings. If the patient devices 200 detect values (e.g., strains, loads, forces, pressures, etc.) that are outside of an acceptable range, the patient devices 200 move to another configuration to keep the detected values within the target range, thereby limiting or avoiding unwanted states or conditions.

If a threshold correction cannot be achieved, a notification can be transmitted to a remote device to alert the patient and/or healthcare provider. Additional treatments or surgical procedures can then be performed. In some embodiments, the patient devices 200 are programmed with one or more target anatomical configurations, such a spine curvature, vertebral spacing, etc. The patient devices 200 can communicate with each other to monitor the current anatomical configuration of the patient. If the anatomical configuration is outside an acceptable range or above/below a predetermined value, the patient devices 200 can automatically move anatomical features (e.g., vertebral bodies) to an acceptable configuration for height restoration, angle correction (e.g., lordosis angle correction, coronal angle correction, etc.), or the like.

The difference between the native anatomical configuration and the corrected anatomical configuration may be referred to as a “patient-specific correction” or “target correction.” The embodiments, features, systems, devices, materials, methods, and techniques described herein may, in certain embodiments, be applied to or used in connection with any one or more of the embodiments, features, systems, devices, or other matter. In some embodiments, mathematical rules defining patient-specific corrections, optimal anatomical outcomes (e.g., positional relationships between anatomic elements) and/or post-operative metrics/design criteria (e.g., adjusted anatomies are used so that post-operative metrics are within an acceptable range for a patient state (e.g., laying down, standing vertical, etc.). Target post-operative metrics can include, but are not limited to, target coronal parameters, target sagittal parameters, target pelvic incidence angle, target Cobb angle, target shoulder tilt, target iliolumbar angle, target coronal balance, target lordosis angle, and/or a target intervertebral space height. For example, the sagittal vertical axis can be less than a set value (e.g., 6 mm, 7 mm, 9 mm, etc.) and the post-operative Cobb angle less than a set value (e.g., 8 degrees, 9 degrees, 10 degrees, etc.), etc., when the patient stands vertically.

The implanted patient device 200a can be an artificial disc with sensors 438a, 438b (collectively “sensors 438”) configured to measure pressures, loads, or forces applied by the first vertebra 410 (e.g., a relatively superior vertebra) and a second vertebra 420 (e.g., a relatively inferior vertebra). The patient device 200a can transmit sensor readings from the sensors 438 to the patient device 200b, illustrated as an extendable rod assembly with sensors (e.g., force sensors, pressure sensors, etc.) and a controller 469. The patient device 200b can receive the transmitted signals and in response move to another configuration based on the received signals. In other embodiments, the patient device 200a generates and transmits commands to the patient device 200b. Both patient devices 200a and 200b can expand to increase the height of an intervertebral space 410 to, for example, reduce or eliminate nerve compress while maintaining target characteristics (e.g., curvature, alignment, etc.) of the spine 400. The amount of expansion can be substantially the same (e.g., to maintain current spine curvature along that section of the spine) or substantially different (e.g., to alter the spine curvature along that section of the spine), etc. In some embodiments, the characteristics of the patient device 200a are controlled based on the sensor readings. Such characteristics include compressibility, range of articulation, etc.

The implanted patient devices 200 may have modules or programs that incorporate one or more mathematical rules based on ranges or thresholds for various disease metrics. For example, an intervention timing module may indicate surgical intervention is necessary if one or more disease metrics exceed a predetermined threshold or meet some other criteria. Representative thresholds that indicate adjustment may be necessary include SVA values greater than 7 mm, a mismatch between lumbar lordosis and pelvic incidence greater than 10 degrees, a Cobb angle of greater than 10 degrees, and/or a combination of Cobb angle and LL/PI mismatch greater than 20 degrees. In some embodiments, indications of adjustment can be based on a percentage change over a period of time. For example, if the SVA value increases by 20% over a period of time, the patient devices 200 can reconfigure themselves to lower the SVA value within 10% of a target SVA value. Other threshold values and metrics can be used; the foregoing are provided as examples only and in no way limit the present disclosure. In some embodiments, the foregoing rules can be tailored to specific patient populations (e.g., for males over 50 years of age or females over 40 years of age, etc.). If a particular patient does not exceed the thresholds indicating adjustment is recommended, the implanted patient devices 200 may provide an estimate for when the patient's metrics will exceed one or more thresholds, thereby providing the patient with an estimate of when adjustment may become recommended. The estimations can be transmitted to an external device for viewing.

The present technology may also include a treatment planning module that can identify the optimal type of correction based on the disease progression of the patient. The treatment planning module can be an algorithm, machine learning model, or other software analytical tool trained or otherwise based on a plurality of reference patient data sets, as previously described. The treatment planning module may also incorporate one or more mathematical rules for identifying adjustments to counter or slow disease progression. As a non-limiting example, if a LL/PI mismatch is between f5 degrees and 10 degrees, the treatment planning module may recommend adjustment to lower LL/PI mismatch below 5 degrees.

In some embodiments, the patient device 200 can include patient-specific endplates 415 and a main body 417. The endplates can be configured to engage the vertebral bodies 410 and 420, respectively. The main body 417 can be include electronics (e.g., processors, storage devices, communication devices, etc.), sensors 438a, 438b, and actuation devices (e.g., actuation device 212 of FIG. 2). In some embodiments, an actuation device is fluidically driven and the sensors 438a, 438b are pressure sensors. In embodiments, the actuation device includes scissor mechanisms, screw-drive mechanisms, or the mechanical mechanism, and the sensors 438 are sensors discussed in connection with FIG. 2.

An auxiliary implant 453 is also illustrated and can be patient-specific and includes an extendable/contractable rod 457. Fasteners 459 can couple the rod 457 to the vertebrae 410 and 420. Auxiliary patient-specific implants can include, without limitation, rod and screw systems, interspinous spacers, or other orthopedic implants. In some embodiments, the device 430 can also be used with non-patient-specific devices and implants. The systems disclosed herein can include any number of implants designed for implantation at different locations to provide a specific treatment.

In some procedures, the patient device 200a can be in a collapsed or low-profile configuration between the first and second vertebrae 410, 420. For example, the collapsed device 200a can be inserted manually with surgical navigation or via a surgical robot and then expanded at the implantation site. Once deployed, as described in more detail below, the device 200a can provide one or more adjustments, corrections (e.g., corrections to the alignment of the first and second vertebrae 410, 420, spinal segments, etc.), or the like. FIG. 3B shows the discs 200a fully expanded along a vertical axis, indicated by arrows 437, 439, to hold apart the first and second vertebrae 410, 420. When the discs 200a is implanted, the patient can be laying generally horizontal so that the spine is generally unloaded. After surgery, the patient can stand vertically or perform one or more tasks while the sensor continuously or periodically collects data for adjustment of the patient device 200a. An external device can control data collection, including sampling rates, detection schedules, etc. The system 100 can transmit the data to the remote device or network. The remote device or network can determine one or more settings (e.g., height, stiffness, position of endplates, etc.) based on the received data. The settings are sent to the implants 200 so that the implants move to respective new target configurations. The process can be performed any number of times (e.g., monthly, yearly, etc.) for treatment adjustability.

The implants 200 can be programmed to detect adverse events, such as excessive loading, displacement (e.g., lateral movement), or the like. The implant 200 can automatically control adjustment to compensate for the adverse event. For example, if posterior loading exceeds a threshold value, the implant 200a can command the fixation rod implant 200b to lengthen to reduce the posterior loading on the disc 200a. The number, configurations, and capabilities of the patient devices 200 can be selected based on the treatment. For example, an intervertebral device (e.g., cage, artificial disc, etc.) can be at each level for treatment.

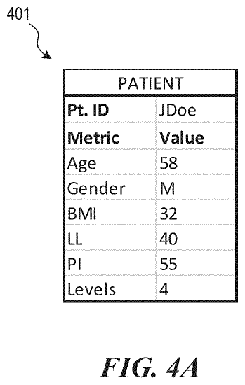

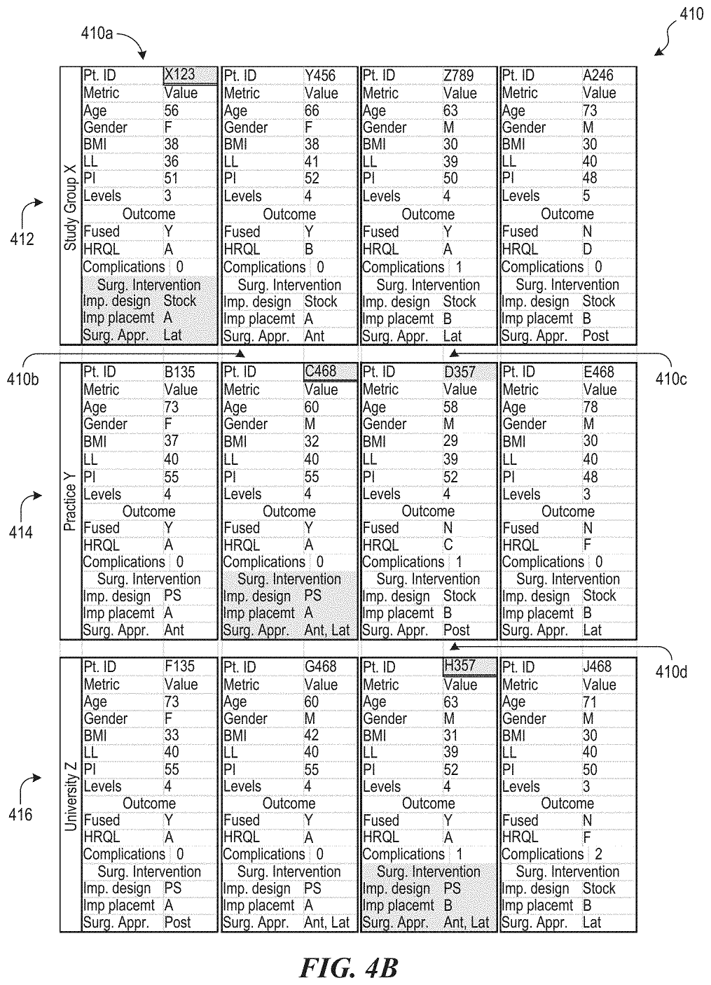

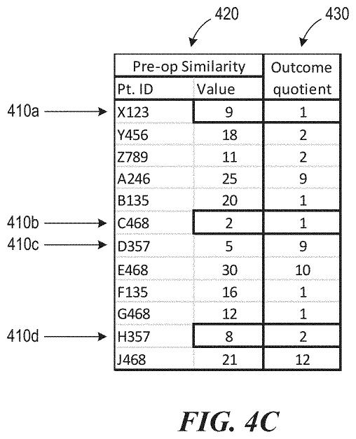

FIGS. 4A-4C illustrate exemplary data sets that may be used and/or generated in connection with the methods described herein, according to an embodiment. FIG. 4A illustrates a patient data set 401 of a patient to be treated. The patient data set 401 can include a patient ID and multiple pre-operative patient metrics (e.g., age, gender, BMI, LL, PI, and treatment levels of the spine (levels)). FIG. 4B illustrates multiple reference patient data sets 410. In the depicted embodiment, the reference patient data sets 410 include a first subset 412 from a study group (Study Group X), a second subset 414 from a practice database (Practice Y), and a third subset 416 from an academic group (University Z). In alternative embodiments, the reference patient data sets 410 can include data from other sources, as previously described herein. Each reference patient data set can include a patient ID, multiple pre-operative patient metrics (e.g., age, gender, BMI, LL, PI, and treatment levels of the spine (levels)), treatment outcome data (Outcome) (e.g., presence of fusion (fused), HRQL, complications), and treatment procedure data (Surg. Intervention) (e.g., implant design, implant placement, surgical approach).

FIG. 4C illustrates comparison of the patient data set 401 to the reference patient data sets 410. The patient data set 401 can be compared to the reference patient data sets 410 to identify one or more similar patient data sets from the reference patient data sets. In some embodiments, the patient metrics from the reference patient data sets 410 are converted to numeric values and compared the patient metrics from the patient data set 401 to calculate a similarity score 420 (“Pre-op Similarity”) for each reference patient data set. Reference patient data sets having a similarity score below a threshold value can be considered to be similar to the patient data set 401. For example, in the depicted embodiment, reference patient data set 410a has a similarity score of 9, reference patient data set 410b has a similarity score of 2, reference patient data set 410c has a similarity score of 5, and reference patient data set 410d has a similarity score of 8. Because each of these scores are below the threshold value of 10, reference patient data sets 410a-d are identified as being similar patient data sets. The reference patient data sets 410a-d can include data sets associated with reference corrective plans. In some embodiments, the data sets can include patient data from reference patients in which anatomy was periodically or continuously corrected over a period of time. For example, reference patients with postoperative data sets similar to the patient can be identified. A subset of those reference patients with corrective procedures matching criteria of corrective procedures for the patient can be identified as reference corrective data sets. The reference corrective plans, reference corrective data sets, and other reference information can be used to generate, at least in part, patient-specific corrective plans. The number, types, and matching criteria can be selected by a user.

The treatment outcome data of the similar patient data sets 410a-d can be analyzed to determine surgical procedures and/or implant designs with the highest probabilities of success. For example, the treatment outcome data for each reference patient data set can be converted to a numerical outcome score 430 (“Outcome Quotient”) representing the likelihood of a favorable outcome. In the depicted embodiment, reference patient data set 410a has an outcome score of 1, reference patient data set 410b has an outcome score of 1, reference patient data set 410c has an outcome score of 9, and reference patient data set 410d has an outcome score of 2. In embodiments where a lower outcome score correlates to a higher likelihood of a favorable outcome, reference patient data sets 410a, 410b, and 410d can be selected. The treatment procedure data from the selected reference patient data sets 410a, 410b, and 410d can then be used to determine at least one surgical procedure (e.g., implant placement, surgical approach) and/or implant design that is likely to produce a favorable outcome for the patient to be treated.

In some embodiments, a method for providing medical care to a patient is provided. The method can include comparing a patient data set to reference data. The patient data set and reference data can include any of the data types described herein. The method can include identifying and/or selecting relevant reference data (e.g., data relevant to treatment of the patient, such as data of similar patients and/or data of similar treatment procedures), using any of the techniques described herein. A treatment plan can be generated based on the selected data, using any of the techniques described herein. The treatment plan can include one or more treatment procedures (e.g., surgical procedures, instructions for procedures, models or other virtual representations of procedures), one or more medical devices (e.g., implanted devices, instruments for delivering devices, surgical kits), or a combination thereof.

In some embodiments, a system for generating a medical treatment plan is provided. The system can compare a patient data set to multiple reference patient data sets, using any of the techniques described herein. A subset of the reference patient data sets can be selected, e.g., based on similarity and/or treatment outcome, or any other technique as described herein. A medical treatment plan can be generated based at least in part on the selected subset, using any of the techniques described herein. The medical treatment plan can include one or more treatment procedures, one or more medical devices, or any of the other aspects of a treatment plan described herein, or combinations thereof.

In further embodiments, a system is configured to use historical patient data. The system can select historical patient data to develop or select a treatment plan, design medical devices, or the like. Historical data can be selected based on one or more similarities between the present patient and prior patients to develop a prescriptive treatment plan designed for desired outcomes. The prescriptive treatment plan can be tailored for the present patient to increase the likelihood of the desired outcome. In some embodiments, the system can analyze and/or select a subset of historical data to generate one or more treatment procedures, one or more medical devices, or a combination thereof. In some embodiments, the system can use subsets of data from one or more groups of prior patients, with favorable outcomes, to produce a reference historical data set used to, for example, design, develop or select the treatment plan, medical devices, or combinations thereof.

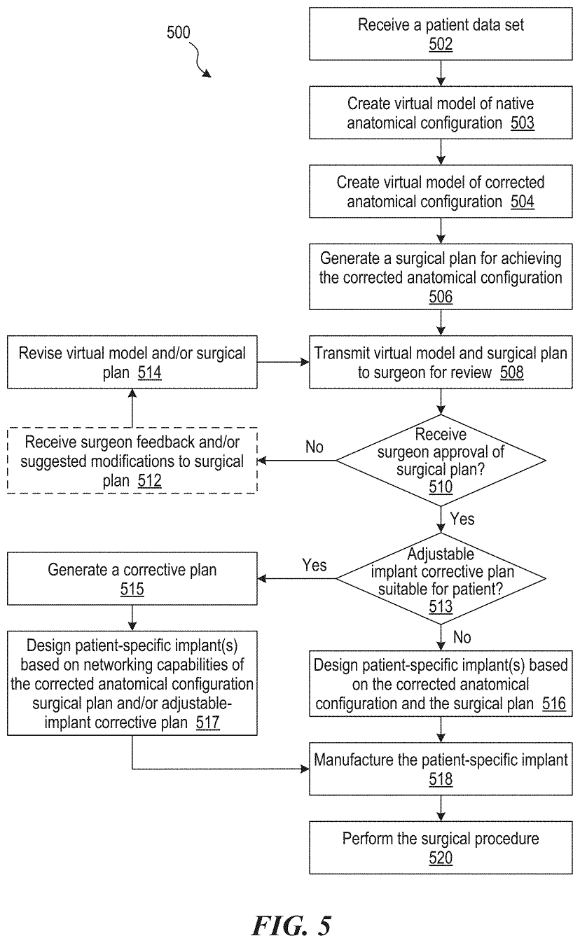

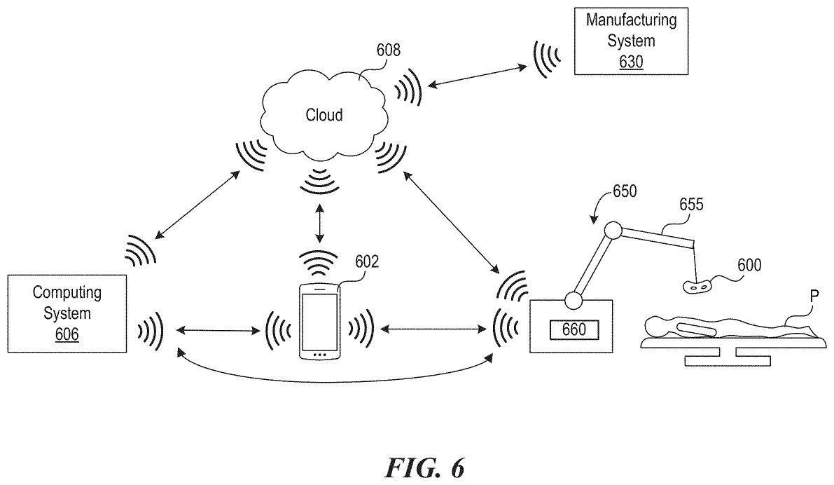

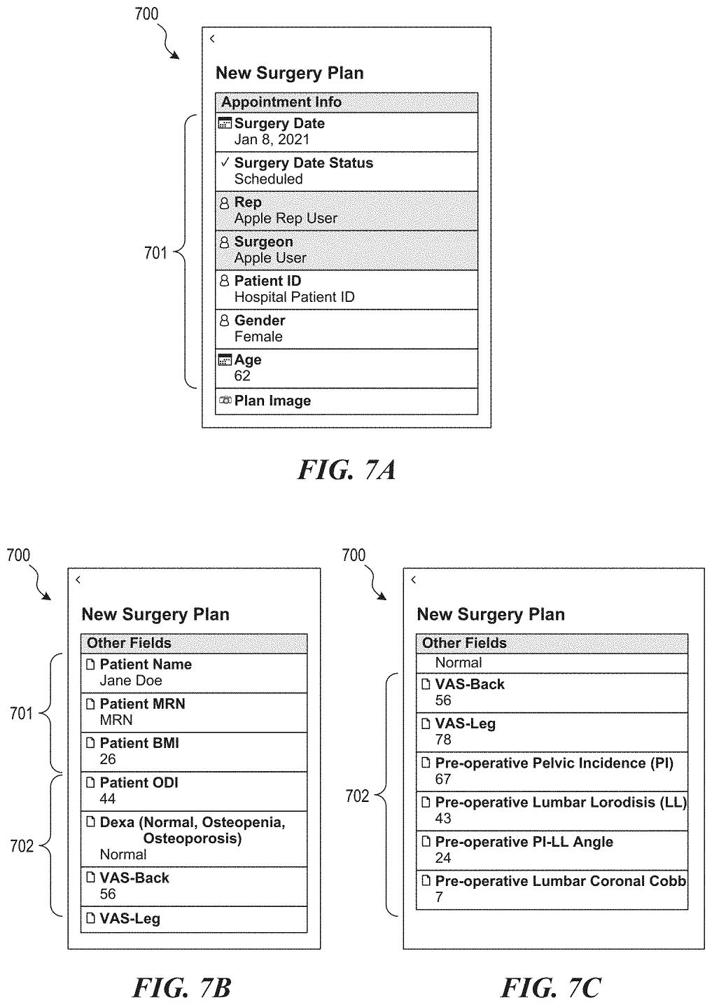

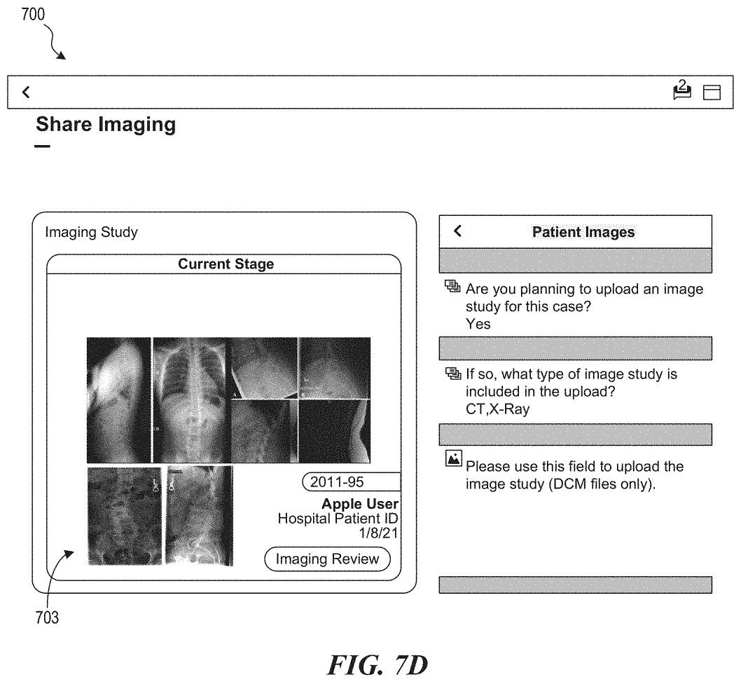

FIG. 5 is a flow diagram illustrating a method 500 for providing patient-specific medical care, according to another embodiment of the present technology. The method 500 can begin in step 502 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, MRI images, ultrasound images, CAT scan images, PET images, X-Ray images, and the like. In some embodiments, the patient data set includes data representing one or more of patient identification number (ID), age, gender, BMI, LL, Cobb angle(s), PI, disc height, segment flexibility, bone quality, rotational displacement, and/or treatment level of the spine. The patient data set can be received at a server, computing device, or other computing system. For example, in some embodiments the patient data set can be received by the server 106 shown in FIG. 1 or the computing system 606 described below with respect to FIG. 6. In some embodiments, the computing system that receives the patient data set in step 502 also stores one or more software modules (e.g., the data analysis module and/or the treatment planning module, or additional software modules for performing various operations of the method 500). Additional details for collecting and receiving the patient data set are described below with respect to FIGS. 6-7D.

In some embodiments, the received patient data set can include disease metrics such as LL, Cobb angles, coronal parameters (e.g., coronal balance, global coronal balance, coronal pelvic tilt, etc.), sagittal parameters (e.g., PI, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 500 includes determining (e.g., automatically determining) one or more of the disease metrics based on the patient image data, as described below.

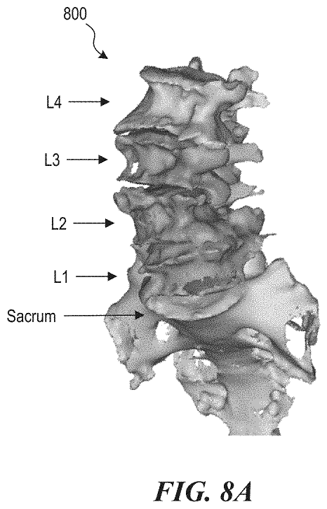

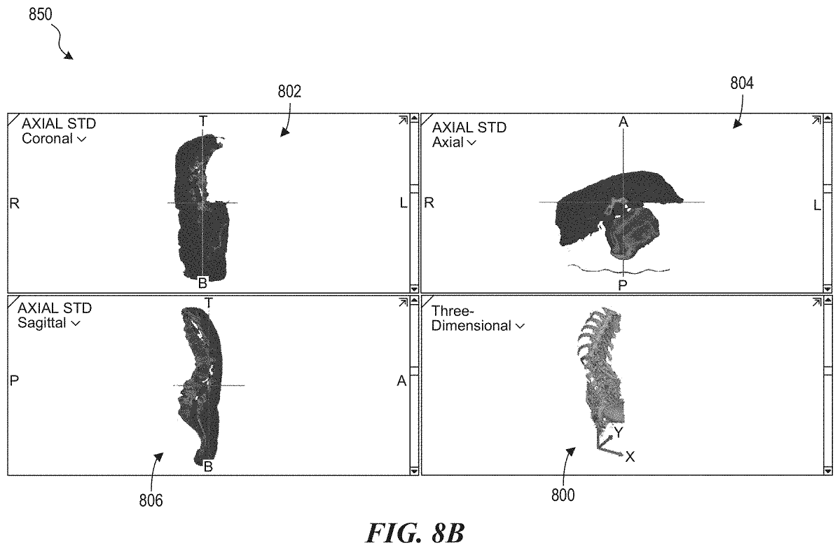

Once the patient data set is received in step 502, the method 500 can continue in step 503 by creating a virtual model of the patient's native anatomical configuration (also referred to as “pre-operative anatomical configuration”). The virtual model can be based on the image data included in the patient data set received in step 502. For example, the same computing system that received the patient data set in step 502 can analyze the image data in the patient data set to generate a virtual model of the patient's native anatomical configuration. The virtual model can be a two- or three-dimensional visual representation of the patient's native anatomy. The virtual model can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 8A and 8B. In some embodiments, the method 500 can optionally omit creating a virtual model of the patient's native anatomy in step 503, and proceed directly from step 502 to step 504.

In some embodiments, the computing system that generated the virtual model in step 502 can also determine (e.g., automatically determine or measure) one or more disease metrics of the patient based on the virtual model. For example, the computing system may analyze the virtual model to determine the patient's pre-operative LL, Cobb angles, coronal parameters (e.g., coronal balance, global coronal balance, coronal pelvic tilt, etc.), sagittal parameters (e.g., PI, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine).

The method 500 can continue in step 504 by creating a virtual model of a corrected anatomical configuration (which can also be referred to herein as the “planned configuration,” “optimized geometry,” “post-operative anatomical configuration,” or “target outcome”) for the patient. For example, the computing system can, using the analysis procedures described previously, determine a “corrected” or “optimized” anatomical configuration for the particular patient that represents an ideal surgical outcome for the particular patient. This can be done, for example, by analyzing multiple reference patient data sets to identify post-operative anatomical configurations for similar patients who had a favorable post-operative outcome, as previously described in detail with respect to FIGS. 1-4C (e.g., based on similarity of the reference patient data set to the patient data set and/or whether the reference patient had a favorable treatment outcome). This may also include applying one or more mathematical rules defining optimal anatomical outcomes (e.g., positional relationships between anatomic elements) and/or target (e.g., acceptable) post-operative metrics/design criteria (e.g., adjust anatomy so that the post-operative sagittal vertical axis is less than 7 mm, the post-operative Cobb angle less than 10 degrees, etc.). Target post-operative metrics can include, but are not limited to, target coronal parameters, target sagittal parameters, target PI angle, target Cobb angle, target shoulder tilt, target iliolumbar angle, target coronal balance, target Cobb angle, target lordosis angle, and/or a target intervertebral space height. The different between the native anatomical configuration and the corrected anatomical configuration may be referred to as a “patient-specific correction” or “target correction.”

Once the corrected anatomical configuration is determined, the computing system can generate a two- or three-dimensional visual representation of the patient's anatomy with the corrected anatomical configuration. As with the virtual model created in step 503, the virtual model of the patient's corrected anatomical configuration can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region in a corrected anatomical configuration, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 9A-1-9B-2.

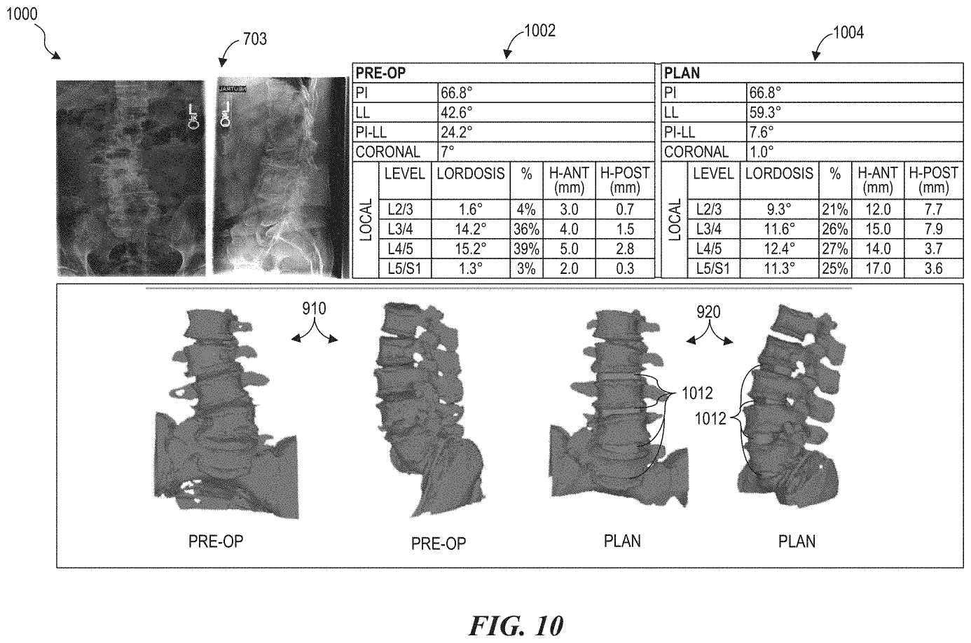

The method 500 can continue in step 506 by generating (e.g., automatically generating) a surgical plan for achieving the corrected anatomical configuration shown by the virtual model. The surgical plan can include pre-operative plans, operative plans, post-operative plans, and/or specific spine metrics associated with the optimal surgical outcome. For example, the surgical plans can include a specific surgical procedure for achieving the corrected anatomical configuration. In the context of spinal surgery, the surgical plan may include a specific fusion surgery (e.g., PLIF, ALIF, TLIF, LLIF, DLIF, XLIF, etc.) across a specific range of vertebral levels (e.g., L1-L4, L1-L5, L3-T12, etc.). Of course, other surgical procedures may be identified for achieving the corrected anatomical configuration, such as non-fusion surgical approaches and orthopedic procedures for other areas of the patient. The surgical plan may also include one or more expected spine metrics (e.g., LL, Cobb angles, coronal parameters, sagittal parameters, and/or pelvic parameters) corresponding to the expected post-operative patient anatomy. The surgical plan can be generated by the same or different computing system that created the virtual model of the corrected anatomical configuration. In some embodiments, the surgical plan can also be based on one or more reference patient data sets as previously described with respect to FIGS. 1-4C. In some embodiments, the surgical plan can also be based at least in part on surgeon-specific preferences and/or outcomes associated with a specific surgeon performing the surgery. In some embodiments, more than one surgical plan is generated in step 506 to provide a surgeon with multiple options. An example of a surgical plan is described below with respect to FIG. 10.

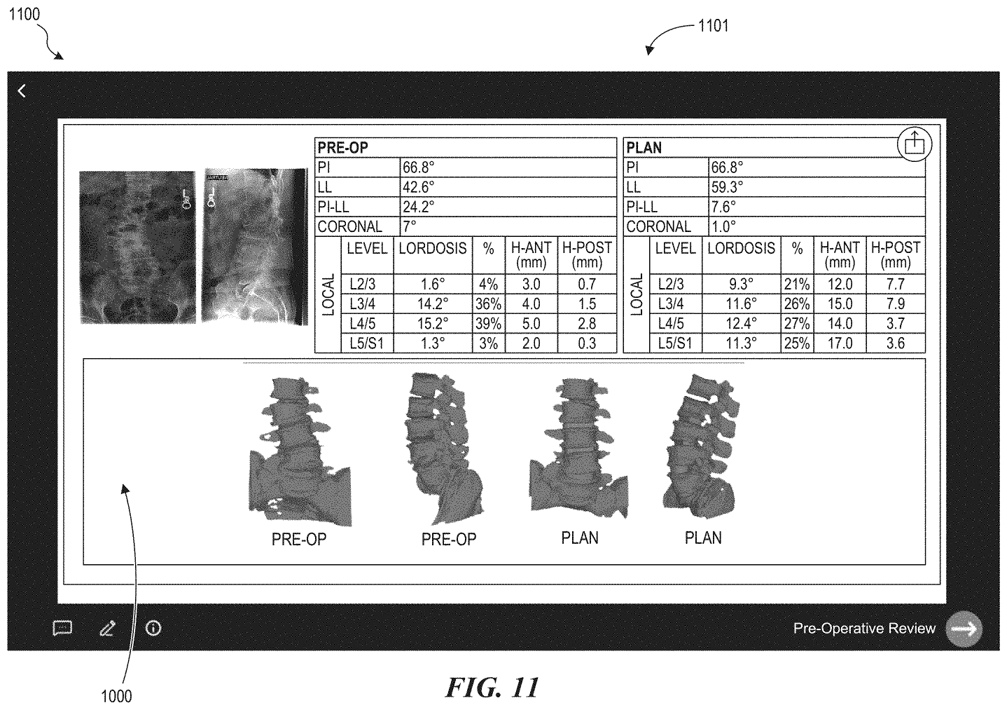

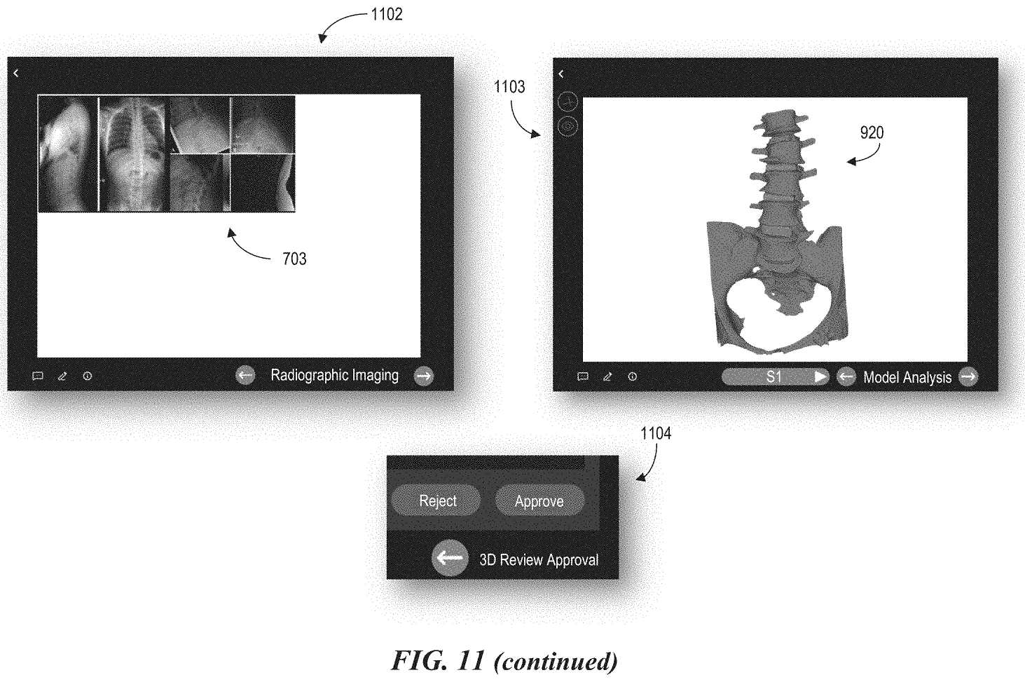

After the virtual model of the corrected anatomical configuration is created in step 504 and the surgical plan is generated in step 506, the method 500 can continue in step 508 by transmitting the virtual model of the corrected anatomical configuration and the surgical plan for surgeon review. In some embodiments, the virtual model and the surgical plan are transmitted as a surgical plan report, an example of which is described with respect to FIG. 11. In some embodiments, the same computing system used in steps 502-506 can transmit the virtual model and surgical plan to a computing device for surgeon review (e.g., the client computing device 102 described in FIG. 1 or the computing device 602 described below with respect to FIG. 6). This can include directly transmitting the virtual model and the surgical plan to the computing device or uploading the virtual model and the surgical plan to a cloud or other storage system for subsequent downloading. Although step 508 describes transmitting the surgical plan and the virtual model to the surgeon, one skilled in the art will appreciate from the disclosure herein that images of the virtual model may be included in the surgical plan transmitted to the surgeon, and that the actual model need not be included (e.g., to decrease the file size being transmitted). Additionally, the information transmitted to the surgeon in step 508 may include the virtual model of the patient's native anatomical configuration (or images thereof) in addition to the virtual model of the corrected anatomical configuration. In embodiments in which more than one surgical plan is generated in step 506, the method 500 can include transmitting more than one surgical plan to the surgeon for review and selection.

The surgeon can review the virtual model and surgical plan and, in step 510, either approve or reject the surgical plan (or, if more than one surgical plan is provided in step 508, select one of the provided surgical plans). If the surgeon does not approve the surgical plan in step 510, the surgeon can optionally provide feedback and/or suggested modifications to the surgical plan (e.g., by adjusting the virtual model or changing one or more aspects about the plan). Accordingly, the method 500 can include receiving (e.g., via the computing system) the surgeon feedback and/or suggested modifications. If surgeon feedback and/or suggested modifications are received in step 512, the method 500 can continue in step 514 by revising (e.g., automatically revising via the computing system) the virtual model and/or surgical plan based at least in part on the surgeon feedback and/or suggested modifications received in step 512. In some embodiments, the surgeon does not provide feedback and/or suggested modifications if they reject the surgical plan. In such embodiments, step 512 can be omitted, and the method 500 can continue in step 514 by revising (e.g., automatically revising via the computing system) the virtual model and/or the surgical plan by selecting new and/or additional reference patient data sets. The revised virtual model and/or surgical plan can then be transmitted to the surgeon for review. Steps 508, 510, 512, and 514 can be repeated as many times as necessary until the surgeon approves the surgical plan. Although described as the surgeon reviewing, modifying, approving, and/or rejecting the surgical plan, in some embodiments the surgeon can also review, modify, approve, and/or reject the corrected anatomical configuration shown via the virtual model.

Once surgeon approval of the surgical plan is received in step 510, the method 500 can continue to step 513 by determining whether a suitable adjustable-implant corrective plan (“corrective plan”) for providing one or more post-operative implant adjustments can be generated based on the approved surgical plan. For example, the computing system can perform one or more simulations in which one or more implants perform post-operative adjustments designed to achieve simulated outcomes (e.g., outcomes of the surgical plan, corrective plan, etc.). If the simulated outcomes meet suitability criteria (e.g., user inputted criteria, threshold metrics from the surgical plan, etc.), the computing system can determine that the corrective plan is suitable for the surgical plan. In some embodiments, the computing system can request modification to the surgical plan in order to generate corrective plans for the modified surgical plan. This provides flexibility to coordinate surgical plans and adjustable implant corrective plans.

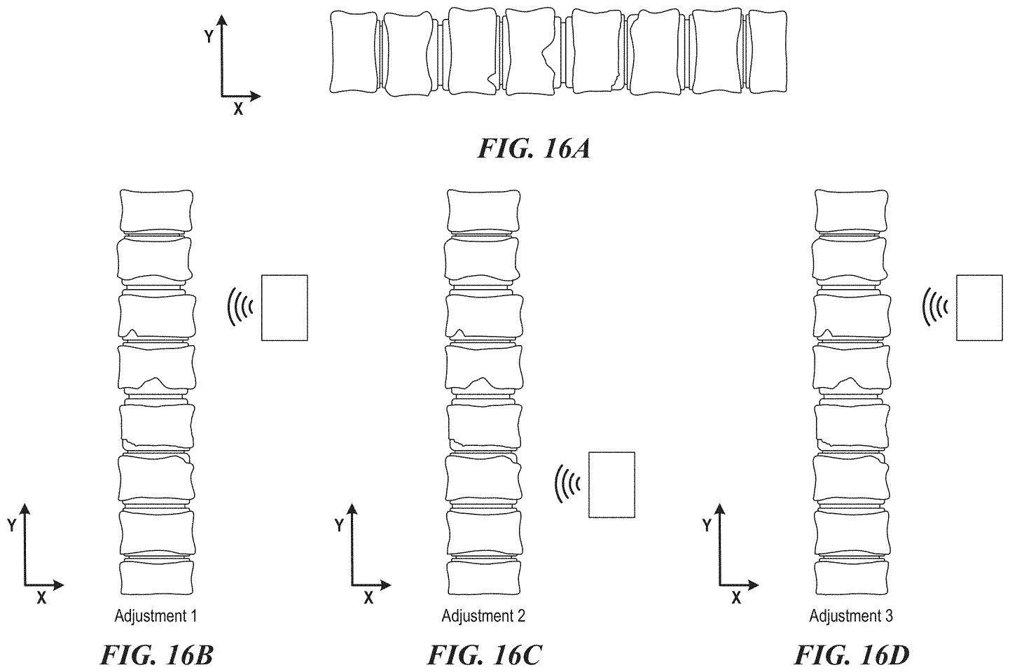

If the computing system determines that a suitable corrective plan can be generated, the method 500 can continue to step 515. The computing system can analyze, for example, one or more surgical plans, designs of adjustable implants, selectable sensor readings, and/or design parameters to generate a corrective plan that meets one or more outcome criteria. In some embodiments, reference data sets can be used to determine a patient-specific post-operative corrective plan. For example, step 513 can include one or more acts of step 316 of FIG. 3A and select a subset of reference data sets with adjustable-implant data based on similarity criteria. This allows the computing system to use reference data that meets criteria for designing implants (e.g., non-adjustable implants, non-adjustable implants, etc.). In some embodiments, the physician can input design parameters, including, without limitation, ranges or values for disk heights, anterior disk heights, posterior disk heights, lordosis angles, and other parameters disclosed herein. Example design parameters for adjustable implants and physician approval are discussed in connection with FIG. 16B and can be used to generate patient-specific post-operative corrective plans in the form of corrective plans (e.g., implant specific corrective plans, multi-implant corrective plans, etc.) used to perform single level or multi-level post-operative spinal adjustments.

In some embodiments, the computing system can generate a corrective plan based on physician inputted design parameters by, for example, selecting levels for treatment, number of implants (e.g., per level, per spinal segment, etc.), and designs for the respective implants. The corrective plan can include post-operative adjustments that can be, for example, incorporated into post-operative therapy or other plans disclosed herein. In some embodiments, the computing system can determine, for example, types of adjustable implants (e.g., expandable disks, rods, expandable cages, etc.), range of motion for individual implants, sensing capabilities of implants, or the like based on computer generated simulations using, for example, three-dimensional models of the patient's anatomy. The physician can approve or revise the corrective plan.

The method 500 can proceed to step 517 to design (e.g., via the same computing system that performed one or more of steps 502-514) patient-specific implant(s) based on networking capabilities, the surgical plan, and/or the corrective plan. The networking capabilities can include, without limitation, one or more of communication ranges, communication frequencies, power settings (e.g., power consumption, power management, etc.), messaging formation, networking protocols, or a combination thereof. The networking capabilities can be selected based on, for example, positions (e.g., implantation sites, carrying sites, etc.) of the patient-devices, planned service life, and/or other parameters disclosed herein. The patient-specific implant(s) can have post-implantation configurations for achieving targeted corrections, such as corrections in the surgical plan. The adjustability of the patient-specific implants can then be designed based on the corrective plan, which can include, without limitation, one or more implant configurations (e.g., undeployed or collapsed configurations, partially deployed or expanded configurations, fully deployed or fully expanded configurations), ranges of motion, relationships between implants (e.g., configuration relationships, loading relationships, etc.), sensing capabilities, or the like. The implantable patient-devices can communicate with each other to coordinate positioning of one or more anatomical elements of the patient. In some embodiments, the computer system can, for example, simulate disease progressions, natural age-related anatomical changes, target spinal configurations, or other anatomical changes to design implants capable of achieving outcomes via post-operative in-vivo adjustments. In some embodiments, the computer system can simulate multiple scenarios of anatomical changes. The scenarios can be ranked to prioritize and/or select design parameters. The system can request physician input to, for example, select the rankings. In some embodiments, the computer system can score and rank the scenarios. The highest-ranking scenario can be selected to design implants that meet a confidence score threshold.

The method 500 can continue in step 518 by manufacturing the patient-specific implant. The implant can be manufactured using additive manufacturing techniques, such as 3D printing, stereolithography, DLP, FDM, SLS, SLM, EBM, LOM, PP, thermoplastic printing, DMD, or inkjet photo resin printing, or like technologies, or combination thereof. Alternatively or additionally, the implant can be manufactured using subtractive manufacturing techniques, such as CNC machining, EDM, grinding, laser cutting, water jet machining, manual machining (e.g., milling, lathe/turning), or like technologies, or combinations thereof. Electronic device(s) can be incorporated into the implant during and/or after forming the structural bearing components of implants. The implant may be manufactured by any suitable manufacturing system (e.g., the manufacturing system 630 described below with respect to FIG. 6). In some embodiments, the implant is manufactured by the manufacturing system executing the computer-readable fabrication instructions generated by the computing system in step 516.

Once the implant is manufactured in step 518, the method 500 can continue in step 520 by implanting the patient-specific implant into the patient. The surgical procedure can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure. Additional details regarding a robotic surgical platform are described below with respect to FIG. 6.

At step 512, if the suitable corrective plan is not identified for the approved surgical plan, the method 500 can proceed to step 516 to design (e.g., via the same computing system that performed steps 502-514) patient-specific implant(s) based on the corrected anatomical configuration and the surgical plan. For example, the patient-specific implant can be specifically designed such that, when it is implanted in the particular patient, it directs the patient's anatomy to occupy the corrected anatomical configuration (e.g., transforming the patient's anatomy from the native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

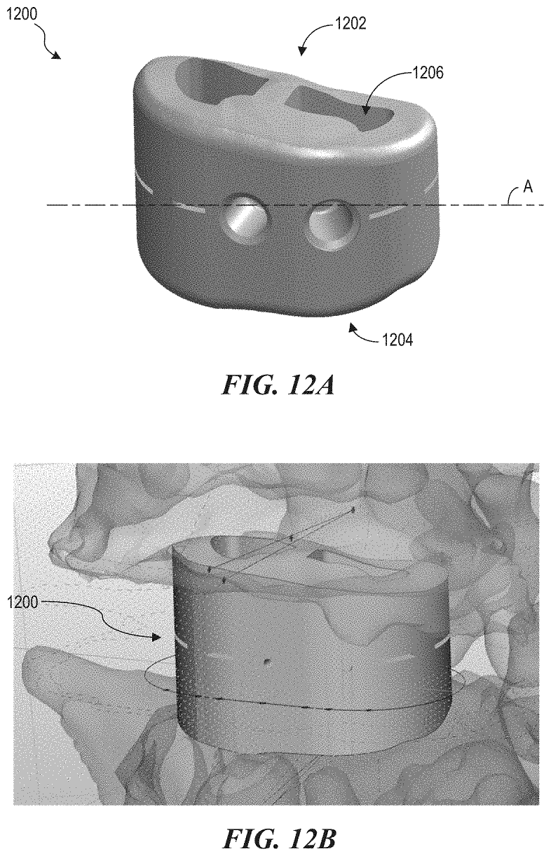

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). An example of a patient-specific implant designed via the method 500 is described below with respect to FIGS. 12A and 12B.

In some embodiments, designing the implant in step 516 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed at step 516 only after the surgeon has reviewed and approved the virtual model with the corrected anatomical configuration and the surgical plan. Accordingly, in some embodiments, the implant design is neither transmitted to the surgeon with the surgical plan in step 508, nor manufactured before receiving surgeon approval of the surgical plan. Without being bound by theory, waiting to design the patient-specific implant until after the surgeon approves the surgical plan may increase the efficiency of the method 500 and/or reduce the resources necessary to perform the method 500.

The method 500 can then proceed to steps 518 and 520 as discussed above. The method 500 can be implemented and performed in various ways. In some embodiments, steps 502-516 can be performed by a computing system (e.g., the computing system 606 described below with respect to FIG. 6) associated with a first entity, step 518 can be performed by a manufacturing system associated with a second entity, and step 520 can be performed by a surgical provider, surgeon, and/or robotic surgical platform associated with a third entity. Any of the foregoing steps may also be implemented as computer-readable instructions stored in memory and executable by one or more processors of the associated computing system(s).

FIG. 6 is a schematic illustration of an operative setup including select systems and devices that can be used to provide patient-specific medical care, such as for performing the method 500 described with respect to FIG. 5. As shown, the operative setup includes a computing device 602, a computing system 606, a cloud 608, a manufacturing system 630, and a robotic surgical platform 650. The computing device 602 can be a user device, such as a smart phone, mobile device, laptop, desktop, personal computer, tablet, phablet, or other such devices known in the art. In operation, a user (e.g., a surgeon) can collect, retrieve, review, modify, or otherwise interact with a patient data set using the computing device 602. The computing system 606 can include any suitable computing system configured to store one or more software modules for identifying reference patient data sets, determining patient-specific surgical plans, generating virtual models of patient anatomy, designing patient-specific implants, or the like. The one or more software modules can include algorithms, machine-learning models, AI architectures, or the like for performing select operations. The cloud 608 can be any suitable network and/or storage system, and may include any combination of hardware and/or virtual computing resources. The manufacturing system 630 can be any suitable manufacturing system for producing patient-specific implants, including any of those previously described herein. The robotic surgical platform 650 (referred to herein as “the platform 650”) can be configured to perform or otherwise assist with one or more aspects of a surgical procedure.

In a representative operation, the computing device 602, the computing system 606, the cloud 608, the manufacturing system 630, and the platform 650 can be used to provide patient-specific medical care, such as to perform the method 500 described with respect to FIG. 5. For example, the computing system 606 can receive a patient data set from the computing device 602 (e.g., step 502 of the method 500). In some embodiments, the computing device 602 can directly transmit, for example, the patient data set, corrective plan, corrective data sets, implant configurations, algorithms (e.g., algorithms for analyzing sensor data, reference data sets, etc.), and other data or information to the computing system 606. In other embodiments, the computing device 602 can upload the patient data set into the cloud 608, and the computing system 606 can download or otherwise access the patient data set from the cloud. Once the computing system 606 receives the data or information (e.g., patient data set), the computing system 606 can create a virtual model of the patient's native anatomical configuration (e.g., step 503 of the method 500), create a virtual model of the corrected anatomical configuration (e.g., step 504 of the method 500), and/or generate a surgical plan for achieving the corrected anatomical configuration (e.g., step 506 of the method 500). The computing system can perform the foregoing operations via the one or more software modules, which in some embodiments include machine learning models or other AI architectures. Once the virtual models and the surgical plan are created, the computing system 606 can transmit the virtual models (e.g., pre-operative models, post-operative models, corrective models, adjustment models, adjustable implant models, etc.) and the plan(s) (e.g., surgical plans, corrective plans, or other plans) to the surgeon for review (e.g., step 508 of the method 500). This can include, for example, directly transmitting the virtual models and the plan(s) to the computing device 602 for surgeon review. In other embodiments, this can include uploading the virtual models and the plan to the cloud 608. A surgeon can then download or otherwise access the virtual models and the plan from the cloud 608 using the computing device 602. The plans can be surgical plans discussed in connection with FIGS. 10-11, corrective plans discussed in connection with FIG. 16B, and other plans disclosed herein.

The surgeon can use the computing device 602 to review the virtual models and the surgical plan. The surgeon can also approve or reject the surgical plan and provide any feedback regarding the surgical plan using the computing device 602. The surgeon's approval, rejection, and/or feedback regarding the surgical plan can be transmitted to, and received by, the computing system 606 (e.g., steps 510 and 512 of the method 500). The computing system 606 can than revise the virtual model and/or the surgical plan (e.g., step 514 of the method 500). The computing system 606 can transmit the revised virtual model and surgical plan to the surgeon for review (e.g., by uploading it to the cloud 608 or directly transmitting it to the computing device 602).

The computing system 606 can also design the patient-specific implant based on the corrected anatomical configuration and the surgical plan (e.g., step 516 of the method 500) using, the one or more software modules. In some embodiments, software modules rely on one or more algorithms, machine learning models, or other AI architectures to design the implant. Once the computing system 606 designs the patient-specific implant, the computing system 606 can upload the design and/or manufacturing instructions to the cloud 608. The computing system 606 may also create fabrication instructions (e.g., computer-readable fabrication instructions) for manufacturing the patient-specific implant. In such embodiments, the computing system 606 can upload the fabrication instructions to the cloud 608.

The manufacturing system 630 can download or otherwise access the design and/or fabrication instructions for the patient-specific implant from the cloud 608. The manufacturing system can then manufacture the patient-specific implant (e.g., step 518 in the method 500) using additive manufacturing techniques, subtractive manufacturing techniques, or other suitable manufacturing techniques.

The robotic surgical platform 650 can perform or otherwise assist with one or more aspects of the surgical procedure (e.g., step 520 of the method 500). For example, the platform 650 can prepare tissue for an incision, make an incision, make a resection, remove tissue, manipulate tissue, perform a corrective maneuver, deliver the implant to a target site, deploy the implant at the target site, adjust the implant at the target site, manipulate the implant once it is implanted, secure the implant at the target site, explant the implant, suture tissue, etc. The platform 650 can therefore include one or more arms 655 and end effectors for holding various surgical tools (e.g., graspers, clips, needles, needle drivers, irrigation tools, suction tools, staplers, screw driver assemblies, etc.), imaging instruments (e.g., cameras, sensors, etc.), and/or medical devices (e.g., the implant 600) and that enable the platform 650 to perform the one or more aspects of the surgical plan. Although shown as having one arm 655, one skilled in the art will appreciate that the platform 650 can have multiple arms (e.g., two, three, four, or more) and any number of joints, linkages, motors, and degrees of freedom. In some embodiments, the platform 650 may have a first arm dedicated to holding one or more imaging instruments, while the remainder of the arms hold various surgical tools. In some embodiments, the tools can be releasably secured to the arms such that they can be selectively interchanged before, during, or after an operative procedure. The arms can be moveable through a variety of ranges of motion (e.g., degrees of freedom) to provide adequate dexterity for performing various aspects of the operative procedure.

The platform 650 can include a control module 660 for controlling operation of the arm(s) 655. In some embodiments, the control module 660 includes a user input device (not shown) for controlling operation of the arm(s) 655. The user input device can be a joystick, a mouse, a keyboard, a touchscreen, an infrared sensor, a touchpad, a wearable input device, a camera- or image-based input device, a microphone, or other user input devices. A user (e.g., a surgeon) can interact with the user input device to control movement of the arm(s) 655.

In some embodiments, the control module 660 includes one or more processors for executing machine-readable operative instructions that, when executed, automatically control operation of the arm 655 to perform one or more aspects of the surgical procedure. In some embodiments, the control module 660 may receive the machine-readable operative instructions (e.g., from the cloud 608) specifying one or more steps of the surgical procedure that, when executed by the control module 660, cause the platform 650 to perform the one or more steps of the surgical procedure. For example, the machine-readable operative instructions may direct the platform 650 to prepare tissue for an incision, make an incision, make a resection, remove tissue, manipulate tissue, perform a corrective maneuver, deliver the implant 600 to a target site, deploy the implant 600 at the target site, adjust a configuration of the implant 600 at the target site, manipulate the implant 600 once it is implanted, secure the implant 600 at the target site, explant the implant 600, suture tissue, and the like. The operative instructions may therefore include particular instructions for articulating the arm 655 to perform or otherwise aid in the delivery of the patient-specific implant. In some embodiments, the control module 660 wirelessly communicates with the implant 600. The control module 660 can transmit intra-operative plans, post-operative plans (e.g., corrective plans for post operative adjustments), data for plans, firmware, software updates, and other data to the implantable patient-device 600. The platform 650 can program and implant any number of implants 600. In some embodiments, the platform 650 implants preprogrammed implants 600. In other embodiments, the platform 650 can implant programmable implant 600. After implantation, the implant 600 can be programmed or reprogrammed using an external electronic device.

In some embodiments, the platform 650 can generate (e.g., as opposed to simply receiving) the machine-readable operative instructions based on the surgical plan. For example, the surgical plan can include information about the delivery path, tools, and implantation site. The platform 650 can analyze the surgical plan and develop executable operative instructions for performing the patient-specific procedure based on the capabilities (e.g., configuration and number of robotic arms, functionality of end effectors, guidance systems, visualization systems, etc.) of the robotic system. This enables the operative setup shown in FIG. 6 to be compatible with a wide range of different types of robotic surgery systems.

The platform 650 can include one or more communication devices (e.g., components having VLC, WiMAX, LTE, WLAN, IR communication, PSTN, Radio waves, Bluetooth, and/or Wi-Fi operability) for establishing a connection with the cloud 608 and/or the computing device 602 for accessing and/or downloading the surgical plan and/or the machine-readable operative instructions. For example, the cloud 608 can receive a request for a particular surgical plan from the platform 650 and send the plan to the platform 650. Once identified, the cloud 608 can transmit the surgical plan directly to the platform 650 for execution. In some embodiments, the cloud 608 can transmit the surgical plan to one or more intermediate networked devices (e.g., the computing device 602) rather than transmitting the surgical plan directly to the platform 650. A user can review the surgical plan using the computing device 602 before transmitting the surgical plan to the platform 650 for execution. Additional details for identifying, storing, downloading, and accessing patient-specific surgical plans are described in U.S. application Ser. No. 16/990,810, filed Aug. 11, 2020, the disclosure of which is incorporated by reference herein in its entirety.

The platform 650 can include additional components not expressly shown in FIG. 6. For example, in various embodiments the platform 650 may include one or more displays (e.g., LCD display screen, an LED display screen, a projected, holographic, or augmented reality display (e.g., a heads-up display device or a head-mounted device), one or more I/O devices (e.g., a network card, video card, audio card, USB, firewire or other external device, camera, printer, speakers, CD-ROM drive, DVD drive, disk drive, or Blu-Ray device), and/or a memory (e.g., RAM, various caches, CPU registers, ROM, and writable non-volatile memory, such as flash memory, hard drives, floppy disks, CDs, DVDs, magnetic storage devices, tape drives, device buffers, and so forth). In some embodiments, the foregoing components can be generally similar to the like components described in detail with respect to computing device 200 in FIG. 2.

Without being bound by theory, using a robotic surgical platform to perform various aspects of the surgical plans described herein is expected to provide several advantages over conventional operative techniques. For example, use of robotic surgical platforms may improve surgical outcomes and/or shorten recovery times by, for example, decreasing incision size, decreasing blood loss, decreasing a length of time of the operative procedure, increasing the accuracy and precision of the surgery (e.g., the placement of the implant at the target location), and the like. The platform 650 can also avoid or reduce user input errors, e.g., by including one or more scanners for obtaining information from instruments (e.g., instruments with retrieval features), tools, the patient specific implant 600 (e.g., after the implant 600 has been gripped by the arm 655), etc. The platform 650 can confirm use of proper instruments prior to and during the surgical procedure. If the platform 650 identifies an incorrect instrument or tool, an alert can be sent to a user that another instrument or tool should be installed. The user can scan the new instrument to confirm that the instrument is appropriate for the surgical plan. In some embodiments, the surgical plan includes instructions for use, a list of instruments, instrument specifications, replacement instruments, and the like. The platform 650 can perform pre- and post-surgical checking routines based on information from the scanners.

FIGS. 7A-13 further illustrate select aspects of providing patient-specific medical care, e.g., in accordance with the method 500. For example, FIGS. 7A-7D illustrate an example of a patient data set 700 (e.g., as received in step 502 of the method 500). The patient data set 700 can include any of the information previously described with respect to the patient data set. For example, the patient data set 700 includes patient information 701 (e.g., patient identification no., patient MRN, patient name, sex, age, BMI, surgery date, surgeon, etc., shown in FIGS. 7A and 7B), diagnostic information 702 (e.g., Oswestry Disability Index (ODI), VAS-back score, VAS-leg score, Pre-operative pelvic incidence, pre-operative lumbar lordosis, pre-operative PI-LL angle, pre-operative lumbar coronal cobb, etc., shown in FIGS. 7B and 7C), and image data 703 (x-ray, CT, MRI, etc., shown in FIG. 7D). In the illustrated embodiment, the patient data set 700 is collected by a healthcare provider (e.g., a surgeon, a nurse, etc.) using a digital and/or fillable report that can be accessed using a computing device (e.g., the computing device 602 shown in FIG. 6). In some embodiments, the patient data set 700 can be automatically or at least partially automatically generated based on digital medical records of the patient. Regardless, once collected, the patient data set 700 can be transmitted to the computing system configured to generate the surgical plan for the patient (e.g., the computing system 606 shown in FIG. 6).

FIGS. 8A and 8B illustrate an example of a virtual model 800 of a patient's native anatomical configuration (e.g., as created in step 503 of the method 500). In particular, FIG. 8A is an enlarged view of the virtual model 800 of the patient's native anatomy and shows the patient's native anatomy of their lower spinal cord region. The virtual model 800 is a three-dimensional visual representation of the patient's native anatomy. In the illustrated embodiment, the virtual model includes a portion of the spinal column extending from the sacrum to the L4 vertebral level. Of course, the virtual model can include other regions of the patient's spinal column, including cervical vertebrae, thoracic vertebrae, lumbar vertebrae, and the sacrum. The illustrated virtual model 800 only includes bony structures of the patient's anatomy, but in other embodiments may include additional structures, such as cartilage, soft tissue, vascular tissue, nervous tissue, etc.

FIG. 8B illustrates a virtual model display 850 (sometimes referred to herein as the “display 850”) showing different views of the virtual model 800. The virtual model display 850 includes a three-dimensional view of the virtual model 800, one or more coronal cross-section(s) 802 of the virtual model 800, one or more axial cross section(s) 804 of the virtual model 800, and/or one or more sagittal cross-section(s) 806 of the virtual model 800. Of course, other views are possible and can be included on the virtual model display 850. In some embodiments, the virtual model 800 may be interactive such that a user can manipulate the orientation or view of the virtual model 800 (e.g., rotate), change the depth of the displayed cross-sections, select and isolate specific bony structures, or the like.

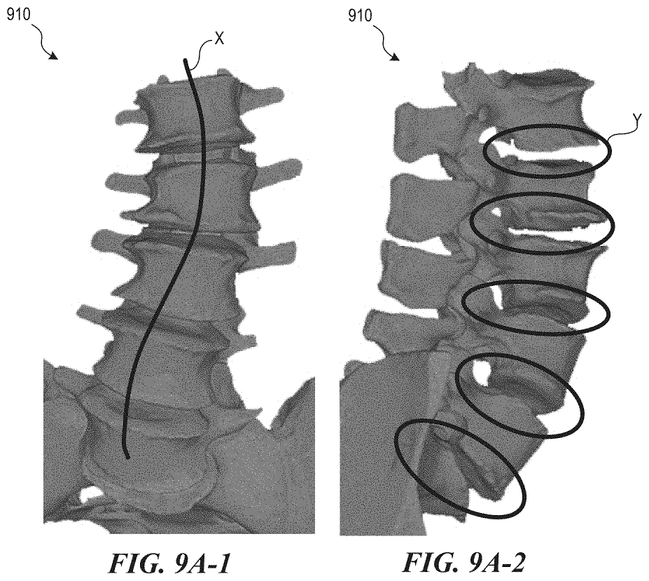

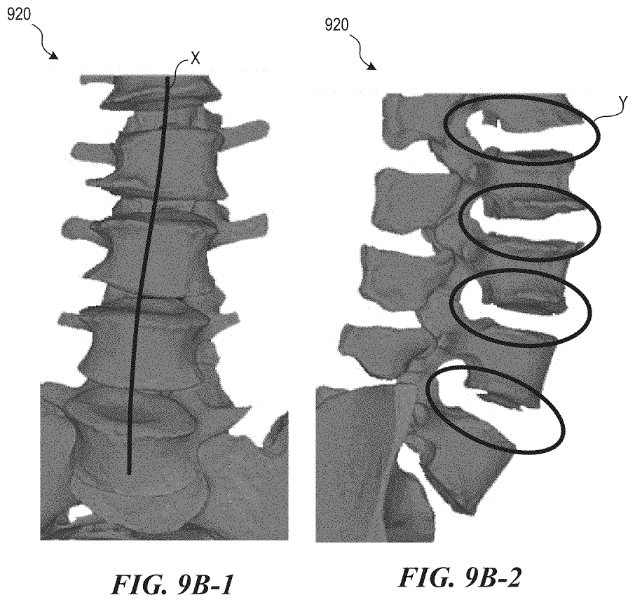

FIGS. 9A-1-9B-2 demonstrate an example of a virtual model of a patient's native anatomical configuration (e.g., as created in step 503 of the method 500) and a virtual model of the patient's corrected anatomical configuration (e.g., as created in step 504 of the method 500). In particular, FIGS. 9A-1 and 9A-2 are anterior and lateral views, respectively, of a virtual model 910 showing a native anatomical configuration of a patient, and FIGS. 9B-1 and 9B-2 are anterior and lateral views, respectively, of a virtual model 920 showing the corrected anatomical configuration for the same patient. Referring first to FIG. 9A-1, the anterior view of the virtual model 910 illustrates the patient has abnormal curvature (e.g., scoliosis) of their spinal column. This is marked by line X, which follows a rostral-caudal axis of the spinal column. Referring next to FIG. 9A-1, the lateral view of the virtual model 910 illustrates the patient has collapsed discs or decreased spacing between adjacent vertebral endplates, marked by ovals Y. FIGS. 9B-1 and 9B-2 illustrate the corrected virtual model 920 accounting for the abnormal anatomical configurations shown in FIGS. 9A-1 and 9A-2. For example, FIG. 9B-1, which is an anterior view of the virtual model 920, illustrates the patient's spinal column having corrected alignment (e.g., the abnormal curvature has been reduced). This correction is shown by line X, which also follows a rostral-caudal axis of the spinal column. FIG. 9B-2, which is a lateral view of the virtual model 920, illustrates the patient's spinal column having restored disc height (e.g., increased spacing between adjacent vertebral endplates), also marked by ovals Y. The lines X and the ovals Y are provided in FIGS. 9A-1-9B-2 to more clearly demonstrate the correction between the virtual models 910 and 920, and are not necessarily included on the virtual models generated in accordance with the present technology.

FIG. 10 illustrates an example of a surgical plan 1000 (e.g., as generated in step 506 of the method 500). The surgical plan 1000 can include pre-operative patient metrics 1002, predicted post-operative patient metrics 1004, one or more patient images (e.g., the patient images 703 received as part of the patient data set), the virtual model 910 (which can be the model itself or one or more images derived from the model) of the patient's native anatomical configuration (e.g., pre-operative patient anatomy), and/or the virtual model 920 (which can be the model itself or one or more images derived from the model) of the patient's corrected anatomical configuration (e.g., predicted post-operative patient anatomy). The virtual model 920 of the predicted post-operative patient anatomy can optionally include one or more implants 1012 shown as implanted in the patient's spinal cord region to demonstrate how patient anatomy will look following the surgery. Although four implants 1012 are shown in the virtual model 920, the surgical plan 1000 may include more or fewer implants 1012, including one, two, three, five, six, seven, eight, or more implants 1012.

The surgical plan 1000 can include additional information beyond what is illustrated in FIG. 10. For example, the surgical plan 1000 may include pre-operative instructions, operative instructions, and/or post-operative instructions. Operative instructions can include one or more specific procedures to be performed (e.g., PLIF, ALIF, TLIF, LLIF, DLIF, XLIF, etc.) and/or one or more specific targets of the operation (e.g., fusion of vertebral levels L1-L4, anchoring screw to be inserted in lateral surface of L4, etc.). Although the surgical plan 1000 is demonstrated in FIG. 10 as a visual report, the surgical plan 1000 can also be encoded in computer-executable instructions that, when executed by a processor connected to a computing device, cause the surgical plan 1000 to be displayed by the computing device. In some embodiments, the surgical plan 1000 may also include machine-readable operative instructions for carrying out the surgical plan. For example, the surgical plan can include operative instructions for a robotic surgical platform to carry out one or more steps of the surgical plan 1000.

FIG. 11 provides a series of images illustrating an example of a patient surgical plan report 1100 that includes the surgical plan 1000 and that may be transmitted to a surgeon for review and approval (e.g., as transmitted in step 508 of the method 500). The surgical plan report 1100 can include a multi-page report detailing aspects of the surgical plan 1000. For example, the multi-page report may include a first page 1101 demonstrating an overview of the surgical plan 1000 (e.g., as shown in FIG. 10), a second page 1102 illustrating patient images (e.g., such as the patient images 703 received in step 502 and shown in FIG. 7D), a third page 1103 illustrating an enlarged view of the virtual model of the corrected anatomical configuration (e.g., the virtual model 920 shown in FIGS. 9B-1 and 9B-2), and a fourth page 1104 prompting the surgeon to either approve or reject the surgical plan 1000. Of course, additional information about the surgical plan can be presented with the report 1100 in the same or different formats. In some embodiments, if the surgeon rejects the surgical plan 1000, the surgeon can be prompted to provide feedback regarding the aspects of the surgical plan 1000 the surgeon would like adjusted.

The patient surgical plan report 1100 can be presented to the surgeon on a digital display of a computing device (e.g., the client computing device 102 shown in FIG. 1 or the computing device 602 shown in FIG. 6). In some embodiments, the report 1100 is interactive and the surgeon can manipulate various aspects of the report 1100 (e.g., adjust views of the virtual model, zoom-in, zoom-out, annotate, etc.). However, even if the report 1100 is interactive, the surgeon generally cannot directly change the surgical plan 1000. Rather, the surgeon may provide feedback and suggested changes to the surgical plan 1000, which can be sent back to the computing system that generated the surgical plan 1000 for analysis and refinement.

FIG. 12A illustrates an example of a patient-specific implant 1200 (e.g., as designed in step 516 and manufactured in step 518 of the method 500), and FIG. 12B illustrates the implant 1200 implanted in the patient. The implant 1200 can be any orthopedic or other implant specifically designed to induce the patient's body to conform to the previously identified corrected anatomical configuration. In the illustrated embodiment, the implant 1200 is an vertebral interbody device having a first (e.g., upper) surface 1202 configured to engage an inferior endplate surface of a superior vertebral body and a second (e.g., lower) surface 1204 configured to engage a superior endplate surface of an inferior vertebral body. The first surface 1202 can have a patient-specific topography designed to match (e.g., mate with) the topography of the inferior endplate surface of the superior vertebral body to form a generally gapless interface therebetween. Likewise, the second surface 1204 can have a patient-specific topography designed to match or mate with the topography of the superior endplate surface of the inferior vertebral body to form a generally gapless interface therebetween. The implant 1200 may also include a recess 1206 or other feature configured to promote bony ingrowth. Because the implant 1200 is patient-specific and designed to induce a geometric change in the patient, the implant 1200 is not necessarily symmetric, and is often asymmetric. For example, in the illustrated embodiment, the implant 1200 has a non-uniform thickness such that a plane defined by the first surface 1202 is not parallel to a central longitudinal axis A of the implant 1200. Of course, because the implants described herein, including the implant 1200, are patient-specific, the present technology is not limited to any particular implant design or characteristic. Additional features of patient-specific implants that can be designed and manufactured in accordance with the present technology are described in U.S. patent application Ser. Nos. 16/987,113 and 17/100,396, the disclosures of which are incorporated by reference herein in their entireties.

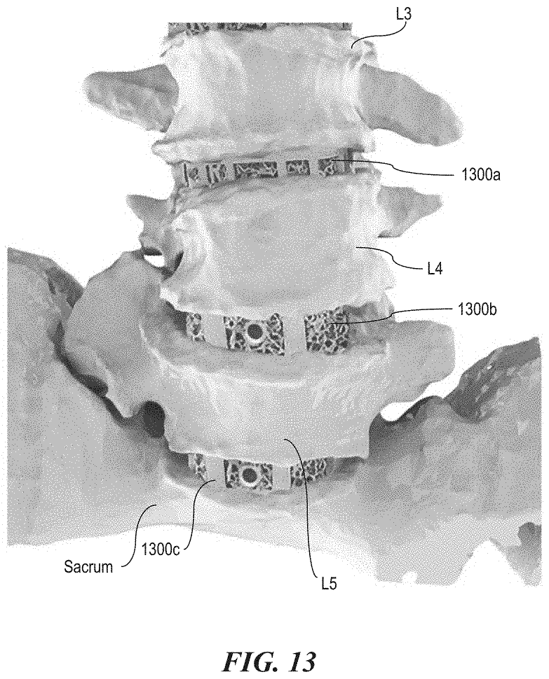

The patient-specific medical procedures described herein can involve implanting more than one patient-specific implant into the patient to achieve the corrected anatomical configuration (e.g., a multi-site procedure). FIG. 13, for example, illustrates a lower spinal cord region having three patient specific implants 1300a-1300c implanted at different vertebral levels. The implants 1300a-1300c can be similar to and include one or more features of the implant discussed in connection with FIGS. 2-3B. More specifically, a first implant 1300a is implanted between the L3 and L4 vertebral bodies, a second implant 1300b is implanted between the L4 and L5 vertebral bodies, and a third implant 1300c is implanted between the L5 vertebral body and the sacrum. Together, the implants 1300a-c can cause the patient's spinal cord region to assume the previously identified corrected anatomical configuration (e.g., transforming the patient's anatomy from its pre-operative diseased configuration to the post-operative optimized configuration). In some embodiments, more or fewer implants are used to achieve the corrected anatomical configuration. For example, in some embodiments one, two, four, five, six, seven, eight, or more implants are used to achieve the corrected anatomical configuration. In embodiments involving more than one implant, the implants do not necessarily have the same shape, size, or function. In fact, the multiple implants will often have different geometries and topographies to correspond to the target vertebral level at which they will be implanted. As also shown in FIG. 13, the patient-specific medical procedures described herein can involve treating the patient at multiple target regions (e.g., multiple vertebral levels).

In addition to designing patient-specific medical care based off reference patient data sets, the systems and methods of the present technology may also design patient-specific medical care based off disease progression for a particular patient. In some embodiments, the present technology therefore includes software modules (e.g., machine learning models or other algorithms) that can be used to analyze, predict, and/or model disease progression for a particular patient. The machine learning models can be trained based off multiple reference patient data sets that includes, in addition to the patient data described with respect to FIG. 1, disease progression metrics for each of the reference patients. The progression metrics can include measurements for disease metrics over a period of time. Suitable metrics may include spinopelvic parameters (e.g., LL, pelvic tilt, sagittal vertical axis (SVA), cobb angle, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.).

In some embodiments, the present technology includes a disease progression module that includes an algorithm, machine learning model, or other software analytical tool for predicting disease progression in a particular patient. The disease progression module can be trained based on reference patient data sets that includes patient information (e.g., age, sex, height, weight, activity level, diet) and disease metrics (e.g., diagnosis, spinopelvic parameters such as LL, pelvic tilt, SVA, cobb angle, coronal offset, etc., disability scores, functional ability scores, flexibility scores, VAS pain scores, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider, who can review and update the estimate, if necessary.

As a non-limiting example, a particular patient who is a fifty-five-year-old male may have an SVA value of 6 mm. The disease progression module can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The systems and methods described herein can also generate models/simulations based on the estimated rates of disease progression, thereby modeling different outcomes over a desired period of time. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical interventions. For example, the disease progression module may simulate what a patient's anatomy may look like 1, 2, 5, or 10 years post-surgery for several surgical intervention options. The simulations may also incorporate non-surgical factors, such as patient age, height, weight, sex, activity level, other health conditions, or the like, as previously described. Based on these simulations, the system and/or a surgeon can select which surgical intervention is best suited for long-term efficacy. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical intervention options or other scenarios. For example, the disease progression module can generate models that predict post-surgical disease progression for each of three different surgical interventions. A surgeon or other healthcare provider can review the disease progression models and, based on the review, select which of the three surgical intervention options is likely to provide the patient with the best long-term outcome. Of course, selecting the optimal intervention can also be fully or semi-automated, as described herein.

Based off of the modeled disease progression, the systems and methods described herein can also (i) identify the optimal time for surgical intervention, and/or (ii) identify the optimal type of surgical procedure for the patient. In some embodiments, the present technology therefore includes an intervention timing module that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' SVA. The data can include (i) SVA values for individual patients over a period of time before surgical intervention (e.g., how fast and to what degree the SVA value changed), (ii) SVA of the individual patients at the time of surgical intervention, (iii) the change in SVA after surgical intervention, and (iv) the degree to which the surgical intervention was successful (e.g., based on pain, quality of life, or other factors). Based on the foregoing data, the intervention timing module can, based on a particular patient's SVA value, identify at which point surgical intervention will have the highest likelihood of producing the most favorable outcome. Of course, the foregoing metric is provided by way of example only, and the intervention timing module can incorporate other metrics (e.g., LL, pelvic tilt, SVA, cobb angle, coronal offset, disability scores, functional ability scores, flexibility scores, VAS pain scores) instead of or in combination with SVA to predict the time at which surgical intervention has the highest probability of providing a favorable outcome for the particular patient.

The intervention timing module may also incorporate one or more mathematical rules based on value thresholds for various disease metrics. For example, the intervention timing module may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only and in no way limit the present disclosure. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module may provide an estimate for when the patient's metrics will exceed one or more thresholds, thereby providing the patient with an estimate of when surgical intervention may become recommended.

The present technology may also include a treatment planning module that can identify the optimal type of surgical procedure for the patient based on the disease progression of the patient. The treatment planning module can be an algorithm, machine learning model, or other software analytical tool trained or otherwise based on multiple reference patient data sets, as previously described. The treatment planning module may also incorporate one or more mathematical rules for identifying surgical procedures. As a non-limiting example, if a LL/PI mismatch is between 10 and 20 degrees, the treatment planning module may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only and in no way limit the present disclosure.

Without being bound by theory, incorporating disease progression modeling into the patient-specific medical procedures described herein may even further increase the effectiveness of the procedures. For example, in many cases it may be disadvantageous operate after a patient's disease progresses to an irreversible or unstable state. However, it may also be disadvantageous to operate too early, before the patient's disease is causing symptoms and/or if the patient's disease may not progress further. The disease progression module and/or the intervention timing module can therefore help identify the window of time during which surgical intervention in a particular patient has the highest probability of providing a favorable outcome for the patient.

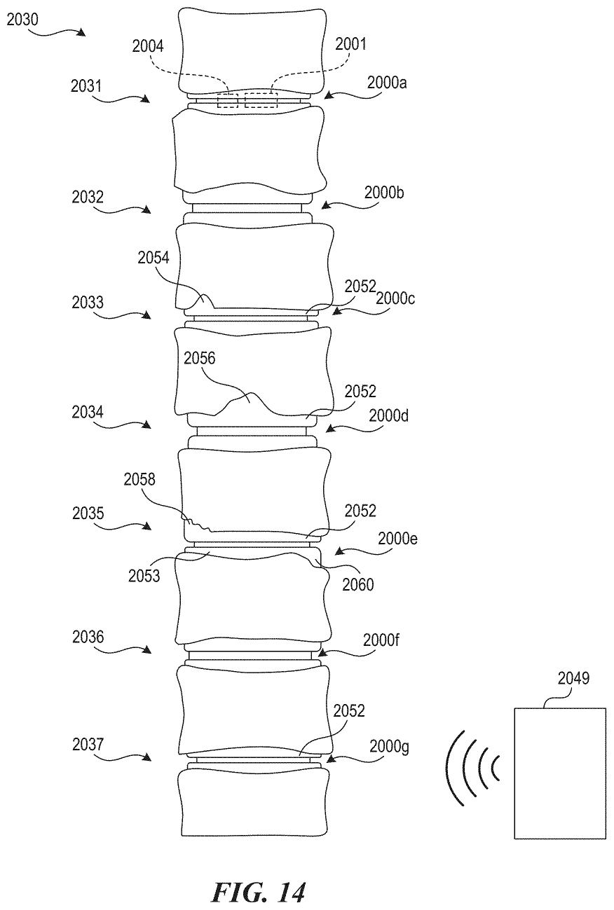

FIG. 14 shows a patient's spine 2030 with intervertebral implants located at each individual level. The implants 2000a-g (collectively, “implants 2000”) can be tailored to fit with anatomical features at the individual levels. Non-invasive post-operative spine adjustments can be performed by using inter-implant communications, a remote device or controller 2049 that controls actuation of the implants 2000, or combinations thereof. The inter-implant communications can be wireless communications via a network maintained by the implants 2000. The remote device 2049 can communicate wirelessly with selected implants or all of the implants. The implants 2000 can be the implants discussed in connection with FIGS. 2-4 or other implants disclosed herein.

An implant 2000a is implanted at a level 2031 with normal endplates free from any defect in the surface topology. The endplates of the implant 2000a can have convex shapes that match the illustrated concave endplates of the adjacent vertebrae at the level 2031. The implant 2000a can have an actuation mechanism 2001 that can be powered by an externally applied field (e.g., magnetic field or another field) provided by the remote device 2049. The actuation mechanism 2001 can include inductively rechargeable power sources, actuation elements, processors, transmitters/receivers, etc. The position, number, and capabilities of the actuation mechanisms (e.g., 2001 or 2004) can be selected based on the available adjustability (e.g., range expansion/contraction, drive force, etc.).

The remote device 2049 can communicate with one or more of the implants 2000. To install new software, the remote device 2049 can wirelessly transmit software to at least one of the implants 2000. The implants 200 can be programmed to wirelessly receive the software, install and run the received software, and/or wirelessly transmit to software to another one of the implants 2000. In some implementations, the remote device 2049 can communicate with the implants 2000 via a wireless protocol, including mesh protocols (e.g., Zigbee protocols, Z-wave protocols, etc.), ad-hoc Network protocols, etc. In some embodiments, the implants 2000 can receive a distribute over the air updates upon authorization. If the implants 2000 detect any adverse event, the implants 2000 can transmit a request for additional software or instructions. The remote device 2049 or another device can receive the request and in response transmit instructions, over-the-air updates, or the like based on the request. In some implementations, the remote device 2049 can be in the form of a smart phone, tablet, or computer that communicates with the implants 2000 via a local wireless connection, such as a Bluetooth connection, Wi-Fi connection, or the like.

The implants 2000 can have patient specific features. An implant 2000b is implanted at a level 2032 with a severe concave shape in the superior and inferior vertebra. The implant 2000b has large convex contours that match the corresponding concave shape of the superior vertebra. An implant 2000c is implanted at a level 2033 with a superior endplate having a focal defect 2054 adjacent, but not on, a longitudinal side of the superior vertebra. The implant 2000d has an upper endplate 2052 with a contouring feature 2056 generally corresponding to the focal defect to better fit the superior endplate. Focal defects in a patient's spine can range from relatively small cavities (e.g., as shown at the level 2033) to relatively large valleys (e.g., as shown at the level 2034). Further, focal defects can include protrusions (not shown) where excess bone and/or cartilage is collected, requiring concave contouring features in the endplates of the implants to match them. An implant 2000e is implanted a level 2035 with corner defects in the superior and inferior vertebrae. Corner defects are located at least partially on longitudinal sides of the vertebrae. Corner defects can include missing corners that are cut off at varying angles, protrusions (not shown) at the corners, and/or rough topology at the corners (e.g., on the missing corner, on the protrusion, and/or on the otherwise normal surface of the corner). The implant 2000e has an upper endplate 2052 with a periphery contour 2058 configured to fit the corner defect in the superior endplate and a lower endplate 2053 with a periphery contour 2060 configured to fit the corner defect in the inferior endplate. Other adjacent levels, such as level 2036, can be formed by endplates with relatively smooth and planar or straight topologies. In such embodiments, an implant 2000f with relatively smooth contouring can be implanted at level 2036.

An implant 2000g is implanted at level 2037 with a superior vertebra having erosive defects on the inferior surface of the superior vertebra. The external device 2049 can command the implant 2000g to move to a target position. As illustrated, erosive defects can span the entire surface of a vertebra and include multiple valleys and peaks therein. In some patients, erosive defects can be contained to a focal region and/or a corner region of a surface. In some patients, erosive defects can include one or more deep valleys and/or one or more tall peaks. As illustrated, the implant 2000g can have an upper endplate 2052 configured to mate with the erosive defects in the superior vertebra.

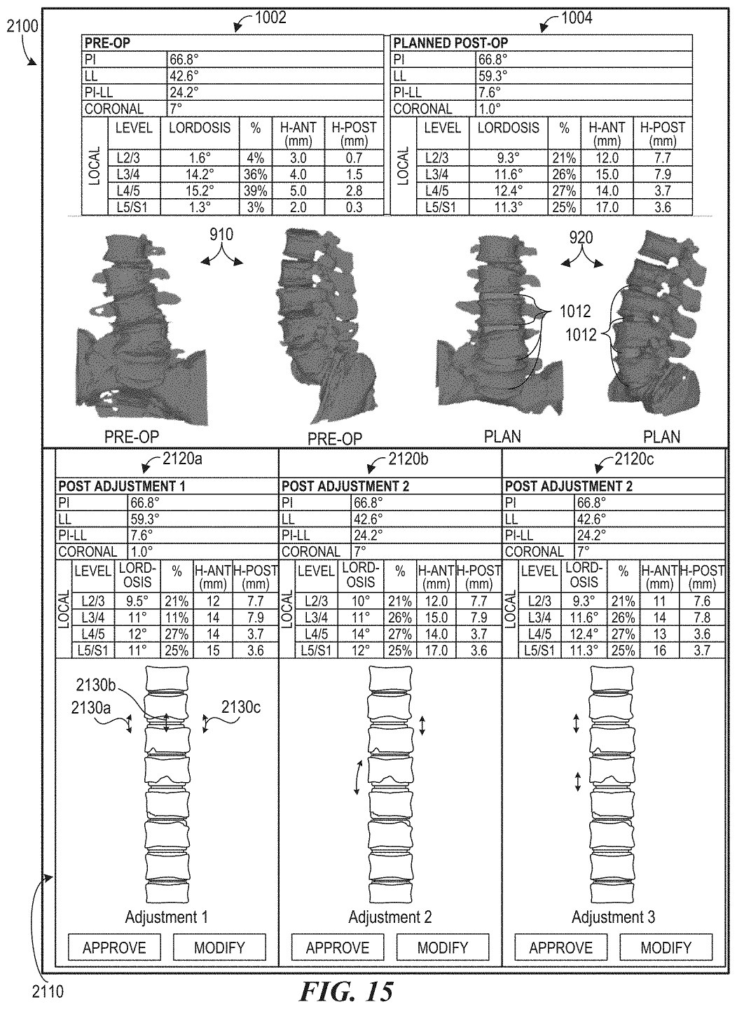

FIG. 15 illustrates an exemplary corrective plan 2100 (e.g., as generated in step 515 of the method 500) for a patient-specific surgical procedure that may be used and/or generated in connection with the methods described herein, according to an embodiment. The corrective plan 2100 can be an adjustable-implant corrective plan that incorporates all or some of surgical plans or other plans disclosed herein. The corrective plan 2100 can include, without limitation, intra- and/or pre-operative patient metrics (e.g., pre-operative patient metrics 1002 discussed in connection with FIGS. 10-13), predicted post-operative patient metrics (e.g., predicted post-operative patient metrics 1004 discussed in connection with FIGS. 10-13), adjustment metrics 2110, and adjustment configurations (e.g., adjustment configurations of implants, adjustment configurations of anatomy, spinal adjustment configurations, etc.).

The adjustment metrics 2110 can include any number of planned adjustments to an adjustable spinal implant. The illustrated corrective plan 2100 includes planned adjustments 2120a, 2120b, 2120c (collectively “adjustments 2120”). Each adjustment 2120 can include associated post-adjustment metrics reviewable by the physician. For example, the physician can review and approve these metrics by selecting an approve button. The computing system can then design the adjustable implants based on the approved adjustment (e.g., design the adjustable implant to have an adjustable range of motion capable of accommodating the approved adjustment(s)). If the physician wants to modify adjustments, the physician can select the modify button. The physician can then input one or more parameters or metrics for adjustment. The computing system can update the spinal model accordingly to the inputted parameters or metrics. Arrows can (e.g., arrows 2130a, 2130b, 2130c) indicate adjustments, such as range of motion, adjustment values, etc. In some environments, the arrows 2130a-b and/or metrics 2120a can indicate degrees of freedom and ranges of motion for specific implants. A user can modify or approve the adjust ability of the implant based on the arrows. Adjustments 2 and 3 include adjustment indicators (illustrated as arrows) showing planned adjustments, such as the adjustments discussed in connection with FIGS. 16A-16D. The physician can approve/select individual target intra-operative configurations and/or post-operative configurations for different loading conditions.

The planned adjustments 2120a, 2120b, 2120c can include configurations for the implantable patient-devices capable of autonomously operating in a coordinate manner to position anatomical elements of the patient to achieve target anatomical configurations. The patient-devices can detect at least one value and then determine an anatomical adjustment based on the detected at least one value and the corrective plan. The values can be inputted by a user or from the corrective plan. One or more of the patient-devices can change configurations to provide the determined anatomical adjustment. The corrective plan (or a portion thereof) 2110 be transmitted to implants. The corrective plan 2110 can include protocols for performing one or more steps of method 300 of FIG. 3A to, for example, discover devices, determined communication details, exchange data, process data, or the like. This allows newly available devices to join the intrabody wireless network. The implants can discover other implants for networking purposes and use predetermined protocols for discovery, as discussed in connection with block 314 in FIG. 3A. For example, if a new device is implanted in or coupled to the patient, another implant can discover the newly available device. The implant can perform the discovery routine of step 314 of FIG. 3A to authenticate and allow the newly available device to join the network.

FIGS. 16A-16D show a patient's spine in different orientations resulting in different loading of implants. FIG. 16A shows the patient's spine in a generally horizontally orientation. For example, the implants can be implanted when the patient's body is generally horizontal so that the spine is generally unloaded. During surgery, it may be difficult to determine how loading of spine will compare to the predicted loading. Accordingly, the implants can be reconfigured post-operatively to move the spine to a target post-operative configuration. FIGS. 16B-16D show adjustment for the post-operative patient's spine in a vertical orientation (e.g., sitting or standing), although post-operative adjustments can also be performed with the patient in other orientations. This allows for post-operative adjustments based on post-operative loading, dynamic visualization, etc. Each of the implants can monitor loading and communicate with other implants to determine, for example, loading along a spinal segment, multi-level loading relationships, and other parameters disclosed herein. Example parameters include intervertebral gap height, lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.), pelvic parameters, or combinations thereof.

In spinal fusion procedures, implants can be adjusted shortly after surgery (e.g., hours, days, etc.) to position at vertebrae for fusion. In spinal alignment procedures, the implants can be vertebral discs adjusted periodically to compensate for patient improvement, disease progression, etc. For example, Adjustments 1-3 of FIGS. 16B-16D can be performed monthly, yearly, or at physician determined intervals. The number of adjustment sessions, period between adjustment sessions, and alterations to the spine can be selected based on a treatment plan, patient recovery, or the like. For example, the system of FIG. 1 and computer device of FIG. 2 can be used to generate correction and adjustments plans. For example, a computer system can be used to determine a corrected anatomical configuration of a patient for achieving a target treatment outcome. The computer system can predict disease progression for a disease affecting the patient's spine based on a patient data set of a patient using at least one machine learning model. The computer system can identify the actuatable implant configured to be implanted in the patient to achieve the corrected anatomical configuration. The actuatable implant is movable between multiple configurations to compensate for the predicted disease progression based on the target treatment outcome. The at least one machine learning model can determine whether to reconfigure the at least one device based on post-adjustment images. The post-adjustment images can include dynamic sit/stand x-ray images, and in some adjustment procedures, the spine can be visualized (e.g., using fluoroscopy) while invasively or non-invasively actuating the actuatable implant.

In some embodiments, one or more anatomical corrections for the patient are generated based on pre-adjustment images and a patient-specific pre-surgical correction plan. A computer system can generate a series of corrected anatomical models representing anatomical changes over a period of time based on a patient-specific correction to the native anatomy and a predicted disease progression. The corrected anatomical models can be viewed and modified by a user as part of the pre-surgical correction plan. The pre-surgical correction plan can be generated by comparing a patient data set to a plurality of reference patient data sets to identify one or more similar patient data sets in the plurality of reference patient data sets, and each similar patient data set corresponds to a reference patient that (a) has similar spinal pathology data as the patient and/or (b) received treatment with an post-operative adjustable orthopedic implant. In some embodiments, a virtual model of the spine is generated. The predicted disease progression using the virtual model. An actuatable implant can be designed to fit the virtual model throughout the predicted disease progression. Simulations can be modified and rerun based on the post-operative adjustments (see FIGS. 16A-16D). Additional implants configured to cooperate with the actuatable implant can be designed to achieve the target treatment outcome and be configured for multi-level adjustments. The plans disclosed herein an provide results (e.g., analytics for each level, overall spine correction score, etc.) from simulations of the multi-level adjustments.

The networked systems and devices disclosed herein can include a data storage element storing patient-specific data, a retrieval feature for accessing patient-specific data, or the like. A data storage module having a memory storing data and a retrieval module configured to transmit the patient-specific surgical plan from the data storage module to a surgical platform can be configured to execute one or more aspects of the patient-specific surgical plan. Patient-specific data is therefore linked to the patient-specific implant. Data can be accessed after the implant is implanted. Data can be used to confirm aspects of the implant/surgery (e.g., is the implant correctly positioned) and be combined, aggregated, and analyzed with post-implantation data (e.g., state of implant data, configuration data, sensor data, etc.). U.S. application Ser. No. 16/990,810 discloses features, systems, devices, materials, and methods that can be incorporated into or used with the networked systems and devices disclosed herein. U.S. application Ser. No. 16/990,810 is incorporated herein by reference in its entirety.

In some embodiments, the present technology can also predict, model, and/or simulate disease progression. For example, a disease progression module may simulate what a patient's anatomy may look like one, two, five, or 10 years post-surgery for several surgical intervention options. The simulations may also incorporate non-surgical factors, such as patient age, height, weight, sex, activity level, other health conditions, or the like, as previously described. Based on these simulations, the system and/or a surgeon can select which surgical intervention is best suited for long-term efficacy. These simulations can also be used to determine patient-specific corrections that compensate for the projected diseases progression. The networked systems and devices can generate data to monitor and predict disease progression. In some embodiments, one or more of the implantable devices includes a disease progression module for local analysis of data. In other embodiments, a remote computing device can include the disease progression module. As the implanted networked systems adjust corrections, the disease progression module can continuously or periodically predict disease progression.

The systems disclosed herein can also include multiple disease progression models (e.g., two, three, four, five, six, or more) that are simulated to provide disease progression data for several different surgical intervention options or other scenarios. For example, the disease progression module can generate models that predict post-surgical disease progression for each of three different surgical interventions. A surgeon or other healthcare provider can review the disease progression models and, based on the review, select which of the three surgical intervention options is likely to provide the patient with the best long-term outcome. Of course, selecting the optimal adjustments can also be fully or semi-automated, as described herein. The implanted networked system can be programmed with the multiple disease progression models. The disease progression models can be modified based on the collected data and healthcare provider, etc.

In some embodiments, networked implants can be used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, or orthopedic trauma. The implants can dynamically correct for irregular spinal curvature, such as scoliosis, lordosis, or kyphosis (hyper- or hypo-), and irregular spinal displacement (e.g., spondylolisthesis). As such, corrections can be varied over time to compensate for disease progress and growth of the patient (e.g., devices implanted when patient is not fully grown, etc.). The networked devices can be designed to treat osteoarthritis, lumbar degenerative disk disease or cervical degenerative disk disease, lumbar spinal stenosis, or cervical spinal stenosis.

The networked systems and devices disclosed herein can include a data storage element storing patient-specific data, a retrieval feature for accessing patient-specific data, or the like. A data storage module having a memory storing data and a retrieval module configured to transmit the patient-specific surgical plan from the data storage module to a surgical platform can be configured to execute one or more aspects of the patient-specific surgical plan. Patient-specific data is therefore linked to the patient-specific implant. Data can be accessed after the implant is implanted. Data can be used to confirm aspects of the implant/surgery (e.g., is the implant correctly positioned) and be combined, aggregated, and analyzed with post-implantation data (e.g., state of implant data, configuration data, sensor data, etc.). U.S. application Ser. No. 16/990,810 discloses features, systems, devices, materials, and methods that can be incorporated into or used with the networked systems and devices disclosed herein. U.S. application Ser. No. 16/990,810 is incorporated herein by reference in its entirety.

In some embodiments, the present technology can also predict, model, and/or simulate disease progression. For example, a disease progression module may simulate what a patient's anatomy may look like one, two, five, or 10 years post-surgery for several surgical intervention options. The simulations may also incorporate non-surgical factors, such as patient age, height, weight, sex, activity level, other health conditions, or the like, as previously described. Based on these simulations, the system and/or a surgeon can select which surgical intervention is best suited for long-term efficacy. These simulations can also be used to determine patient-specific corrections that compensate for the projected diseases progression. The networked systems and devices can generate data to monitor and predict disease progression. In some embodiments, one or more of the implantable devices includes a disease progression module for local analysis of data. In other embodiments, a remote computing device can include the disease progression module. As the implanted networked systems adjust corrections, the disease progression module can continuously or periodically predict disease progression.

The systems disclosed herein can also include multiple disease progression models (e.g., two, three, four, five, six, or more) that are simulated to provide disease progression data for several different surgical intervention options or other scenarios. For example, the disease progression module can generate models that predict post-surgical disease progression for each of three different surgical interventions. A surgeon or other healthcare provider can review the disease progression models and, based on the review, select which of the three surgical intervention options is likely to provide the patient with the best long-term outcome. Of course, selecting the optimal adjustments can also be fully or semi-automated, as described herein. The implanted networked system can be programmed with the multiple disease progression models. The disease progression models can be modified based on the collected data and healthcare provider, etc.

As one skilled in the art will appreciate, any of the software functions described previously may be combined or distributed into one or more software functions or devices for performing the operations described herein. Accordingly, any of the operations described herein can be performed by any of the computing devices or systems described herein, unless expressly noted otherwise.

The networked implants can be used to correct numerous different maladies in a variety of contexts including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. The implants can dynamically correct for irregular spinal curvature, such as scoliosis, lordosis, or kyphosis (hyper- or hypo-), and irregular spinal displacement (e.g., spondylolisthesis). As such, corrections can be varied over time to compensate for disease progress and growth of the patient (e.g., devices implanted when patient is not fully grown, etc.). The networked devices can be designed to treat osteoarthritis, lumbar degenerative disc disease or cervical degenerative disc disease, lumbar spinal stenosis, and cervical spinal stenosis. The networked implants can be orthopedic implants (e.g., artificial hips, fracture repair structures, alignment inserts, spinal-assistance structures, corresponding attachment mechanisms, etc.), sensory/neurological implants, replacement organs, assistive mechanisms (e.g., pacemakers, defibrillators, valves, stents), or the like. Other devices, such as attachable or wearable devices (e.g., blood glucose monitors, heart monitors, etc.), may be attached to or worn on the patient body for significant durations. Still other devices (e.g., personal devices, such as mobile phones, smart watches, and/or other personal health monitors) may be carried by the patient or within a fixed distance from the patient for significant portions of each day. These devices can be part of the network.

### Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A computer-implemented method for establishing and managing a patient-specific device network, the method comprising:
identifying a set of patient-devices using signals with a frequency, a power setting, a messaging formation, or a combination thereof that corresponds to the set of patient-devices being implanted in, attached to, and/or carried on a body of a patient;
determining communication details for exchanging data between the set of patient-devices and/or with an external device; and
communicating data according to the determined communication details for analysis and/or a subsequent corrective action for implementation by a device in the set of patient-devices.

2. The computer-implemented method of example 1, further comprising:
transmitting a treatment plan to at least one of the patient-devices, wherein the treatment plan includes target anatomical corrections to be provided by the set of patient-devices.

3. The computer-implemented method of example 1 and 2, wherein the set of patient-devices are implanted in the patient's body and are programmed for communication with one another to coordinate mechanical adjustments to maintain at least anatomical correction.

4. The computer-implemented method of any of examples 1-3, wherein the set of patient-devices includes:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

5. The computer-implemented method of any of examples 1-4, wherein the set of patient-devices are programmed to autonomously coordinate operation based on wireless communications between the patient-devices to achieve one or more corrections.

6. The computer-implemented method of any of examples 1-5, wherein the set of patient-devices are programmed to reposition vertebrae of the patient's spine to achieve one or more spinal corrections.

7. The computer-implemented method of any of examples 1-6, further comprising:
measuring at least one value using a sensor of the patient-devices;
determining a spinal correction for the patient's spine based on the measured at least one value; and
causing at least one of the patient-devices to move anatomical elements to achieve the determined spinal correction.

8. The computer-implemented method of any of examples 1-7, wherein the at least one value includes one or more of a pressure, a load, or a moment.

9. The computer-implemented method of any of examples 1-8, wherein one or more of the patient-devices has patient-specific interfacing elements.

10. The computer-implemented method of any of examples 1-9, further comprising generating a treatment plan for the patient, wherein the treatment plan includes a schedule for actuating one or more for the patient-devices to keep a correction within a predetermined range.

11. The computer-implemented method of any of examples 1-10, further comprising:
generating a treatment plan for the patient; and
designing the set of patient-devices to achieve the treatment plan, wherein the patient-devices are programmed with the treatment plan.

12. The computer-implemented method of any of examples 1-11, further comprising:
generating data using one or more sensors of the set of patient-devices, and
optimizing a patient-specific treatment provided by the set of patient-devices according to a target treatment outcome.

13. A computer-implemented method for establishing and managing a patient-specific device network, the method comprising:
obtaining sensor readings from a plurality of implanted devices in a subject programmed to wireless communicate with one another;
correlating, using the plurality of implanted devices, operation of one or more of the implanted devices to the sensor readings;
selecting at least one of the implanted devices to be reconfigured based on the correlation and a correction plan for the subject's spine; and
commanding to the selected at least one implanted device to move from a first configuration to a second configuration.

14. The computer-implemented method of any of examples 13, further comprising the second configuration such that the first and second implanted devices together provide one or more correction according to the correction plan.

15. The computer-implemented method of any of examples 13-14, further comprising:
causing two or more of the implanted devices to move to new configurations;
identifying one or more target anatomical changes associated with the new configurations based on sensor measurements acquired by the implanted devices;
using the identified one or more target anatomical changes to correlate the operation of one or more of the implanted devices to the transmitted data.

16. The computer-implemented method of any of examples 13-15, wherein the transmitted data includes sensor measurements.

17. The computer-implemented method of any of examples 13-16, wherein plurality of implanted devices includes a first implanted device and a second implanted device, wherein the first implanted device sends the wirelessly transmitted commands to the second implanted device accordingly to the spinal correction plan.

18. A non-transitory computer-readable storage medium storing instructions that, when executed by a computing system, cause the computing system to perform operations comprising:
identifying a set of patient-devices using signals with a frequency, a power setting, a messaging formation, or a combination thereof that corresponds to the set of patient-devices being implanted in, attached to, and/or carried on a body of a patient;
determining communication details for exchanging data between the set of patient-devices and/or with an external device; and
communicating data according to the determined communication details for analysis and/or a subsequent corrective action for implementation by a device in the set of patient-devices.

19. A system comprising:
two or more patient devices configured to be implanted into, attached to, and/or carried by a human body, wherein each of the two or more patient devices includes:
   one or more processors; and
   a memory storing instructions that, when executed by the one or more processors, cause the system to perform operations comprising:
      identifying a set of patient-devices using signals with a frequency, a power setting, a messaging formation, or a combination thereof that corresponds to the set of patient-devices being implanted in, attached to, and/or carried on a body of a patient;
      determining communication details for exchanging data between the set of patient-devices and/or with an external device; and
      communicating data according to the determined communication details for analysis and/or a subsequent corrective action for implementation by a device in the set of patient-devices.

20. A computer-implemented method for establishing and managing a patient-specific device network, the method comprising:
generating a corrective plan for multiple implantable patient-devices in a patient based on patient data of the patient; and
transmitting data according to the corrective plan to one or more of the implantable patient-devices, wherein the implantable patient-devices are configured to communicate with each other via a wireless mesh network based on the corrective plan.

21. The computer-implemented method of example 20, wherein the implantable patient-devices autonomously operate to coordinate positioning of anatomical elements of the patient according to the corrective plan.

22. The computer-implemented method of any of examples 20-21, wherein the implantable patient-devices are configured to mechanically move anatomical elements of the patient to adjust at least one of lumbar lordosis, Cobb angles, coronal parameters, sagittal parameters, or pelvic parameters according to the corrective plan.

23. The computer-implemented method of any of examples 20-22, further comprising transmitting the corrective plan to at least one of the patient-devices that uses the corrective plan and the transmitted data to provides one or more target anatomical corrections.

24. The computer-implemented method of any of examples 20-23, further comprising transmitting the corrective plan to at least one of the patient-devices, wherein the corrective plan is configured to coordinate operation of one or more of the patient-devices.

25. The computer-implemented method of any of examples 20-24, further comprising:
selecting at least one of the patient-devices based on communication protocols, a power setting, a messaging formation, or a combination.

26. The computer-implemented method of any of examples 20-25, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

27. The computer-implemented method of any of examples 20-26, wherein at least one of the implantable patient-devices is programmed to wirelessly receive software, install and run the received software, and wirelessly transmit to software to another one of the implantable patient devices.

28. The computer-implemented method of any of examples 20-27, further comprising:
generating manufacturing data for the implantable patient devices based on the correct plan; and
manufacturing the implantable patient devices according to the manufacturing data.

29. The computer-implemented method of any of examples 20-28, wherein the multiple implantable patient-devices are programmed to reposition vertebrae of a spine of the patient to achieve one or more spinal corrections according to the corrective plan.

30. The computer-implemented method of any of examples 20-29, further comprising:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

31. The computer-implemented method of any of examples 20-30, further comprising:
identifying a newly available implantable patient-device in the patient;
authenticating the newly available implantable patient-device; and
allowing the authenticated newly available implantable patient-device to become part of the wireless mesh network.

32. The computer-implemented method of any of examples 20-31, further comprising:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

33. The computer-implemented method of any of examples 20-32, wherein the corrective plan includes target ranges of corrections values, wherein the multiple implantable patient-devices are configured to corporate to position anatomical features to keep correction values of the patient within the target ranges of corrections values.

34. The computer-implemented method of any of examples 20-33, wherein the implantable patient-devices include a first implanted device and a second implanted device, wherein the first implanted device sends wirelessly transmitted commands to the second implanted device accordingly to a spinal correction plan.

35. The computer-implemented method of any of examples 20-34, further comprising:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

36. The computer-implemented method of any of examples 20-35, wherein at least of the implantable patient-devices includes a controller programmed to autonomously establish and manage a network among the implanted patient-devices.

37. The computer-implemented method of any of examples 20-36, wherein at least two of the implantable patient-devices are implanted at a level and coordinate operation to maintain one or more spinal corrections.

38. A non-transitory computer-readable storage medium storing instructions that, when executed by a computing system, cause the computing system to perform operations for establishing and managing a patient-specific device network, the operations comprising:
generating a corrective plan for multiple implantable patient-devices in a patient based on patient data of the patient; and
transmitting data according to the corrective plan to one or more of the implantable patient-devices, wherein the implantable patient-devices are configured to communicate with each other via a wireless mesh network based on the corrective plan.

39. The non-transitory computer-readable medium of example 38, wherein the implantable patient-devices autonomously operate to coordinate positioning of anatomical elements of the patient according to the corrective plan.

40. The non-transitory computer-readable medium of any of examples 38-39, wherein the implantable patient-devices are configured to mechanically move anatomical elements of the patient to adjust at least one of lumbar lordosis, Cobb angles, coronal parameters, sagittal parameters, or pelvic parameters according to the corrective plan.

41. The non-transitory computer-readable medium of any of examples 38-40, wherein the operations further comprise transmitting the corrective plan to at least one of the patient-devices that uses the corrective plan and the transmitted data to provides one or more target anatomical corrections.

42. The non-transitory computer-readable medium of any of examples 38-41, wherein the operations further comprise transmitting the corrective plan to at least one of the patient-devices, wherein the corrective plan is configured to coordinate operation of one or more of the patient-devices.

43. The non-transitory computer-readable medium of any of examples 38-42, wherein the operations further comprise:
selecting at least one of the patient-devices based on communication protocols, a power setting, a messaging formation, or a combination.

44. The non-transitory computer-readable medium of any of examples 38-43, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

45. The non-transitory computer-readable medium of any of examples 38-44, wherein at least one of the implantable patient-devices is programmed to wirelessly receive software, install and run the received software, and wirelessly transmit to software to another one of the implantable patient devices.

46. The non-transitory computer-readable medium of any of examples 38-45, wherein the operations further comprise:
generating manufacturing data for the implantable patient devices based on the correct plan; and
manufacturing the implantable patient devices according to the manufacturing data.

47. The non-transitory computer-readable medium of any of examples 38-46, wherein the multiple implantable patient-devices are programmed to reposition vertebrae of a spine of the patient to achieve one or more spinal corrections according to the corrective plan.

48. The non-transitory computer-readable medium of any of examples 38-47, wherein the operations further comprise:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

49. The non-transitory computer-readable medium of any of examples 38-48, wherein the operations further comprise:
identifying a newly available implantable patient-device in the patient;
authenticating the newly available implantable patient-device; and
allowing the authenticated newly available implantable patient-device to become part of the wireless mesh network.

50. The non-transitory computer-readable medium of any of examples 38-49, wherein the operations further comprise:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

51. The non-transitory computer-readable medium of any of examples 38-50, wherein the corrective plan includes target ranges of corrections values, wherein the multiple implantable patient-devices are configured to corporate to position anatomical features to keep correction values of the patient within the target ranges of corrections values.

52. The non-transitory computer-readable medium of any of examples 38-51, wherein the implantable patient-devices include a first implanted device and a second implanted device, wherein the first implanted device sends wirelessly transmitted commands to the second implanted device accordingly to a spinal correction plan.

53. The non-transitory computer-readable medium of any of examples 38-52, wherein the operations further comprise:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

54. The non-transitory computer-readable medium of any of examples 38-53, wherein at least of the implantable patient-devices includes a controller programmed to autonomously establish and manage a network among the implanted patient-devices.

55. The non-transitory computer-readable medium of any of examples 38-54, wherein at least two of the implantable patient-devices are implanted at a level and coordinate operation to maintain one or more spinal corrections.

56. A system comprising:
one or more processors; and
one or more memories storing instructions that, when executed by the one or more processors, cause the system to perform a process for establishing and managing a patient-specific device network, the process comprising:
   generating a corrective plan for multiple implantable patient-devices in a patient based on patient data of the patient; and
   transmitting data according to the corrective plan to one or more of the implantable patient-devices, wherein the implantable patient-devices are configured to communicate with each other via a wireless mesh network based on the corrective plan.

57. The system according to example 56, wherein the implantable patient-devices autonomously operate to coordinate positioning of anatomical elements of the patient according to the corrective plan.

58. The system according to any of examples 56-57, wherein the implantable patient-devices are configured to mechanically move anatomical elements of the patient to adjust at least one of lumbar lordosis, Cobb angles, coronal parameters, sagittal parameters, or pelvic parameters according to the corrective plan.

59. The system according to any of examples 56-58, wherein the process further comprises transmitting the corrective plan to at least one of the patient-devices that uses the corrective plan and the transmitted data to provides one or more target anatomical corrections.

60. The system according to any of examples 56-59, wherein the process further comprises transmitting the corrective plan to at least one of the patient-devices, wherein the corrective plan is configured to coordinate operation of one or more of the patient-devices.

61. The system according to any of examples 56-60, wherein the process further comprises:
selecting at least one of the patient-devices based on communication protocols, a power setting, a messaging formation, or a combination.

62. The system according to any of examples 56-61, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

63. The system according to any of examples 56-62, wherein at least one of the implantable patient-devices is programmed to wirelessly receive software, install and run the received software, and wirelessly transmit to software to another one of the implantable patient devices.

64. The system according to any of examples 56-63, wherein the process further comprises:
generating manufacturing data for the implantable patient devices based on the correct plan; and
manufacturing the implantable patient devices according to the manufacturing data.

65. The system according to any of examples 56-64, wherein the multiple implantable patient-devices are programmed to reposition vertebrae of a spine of the patient to achieve one or more spinal corrections according to the corrective plan.

66. The system according to any of examples 56-65, wherein the process further comprises:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

67. The system according to any of examples 56-66, wherein the process further comprises:
identifying a newly available implantable patient-device in the patient;
authenticating the newly available implantable patient-device; and
allowing the authenticated newly available implantable patient-device to become part of the wireless mesh network.

68. The system according to any of examples 56-67, wherein the process further comprises:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

69. The system according to any of examples 56-68, wherein the corrective plan includes target ranges of corrections values, wherein the multiple implantable patient-devices are configured to corporate to position anatomical features to keep correction values of the patient within the target ranges of corrections values.

70. The system according to any of examples 56-69, wherein the implantable patient-devices include a first implanted device and a second implanted device, wherein the first implanted device sends wirelessly transmitted commands to the second implanted device accordingly to a spinal correction plan.

71. The system according to any of examples 56-70, wherein the process further comprises:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

72. The system according to any of examples 56-71, wherein at least of the implantable patient-devices includes a controller programmed to autonomously establish and manage a network among the implanted patient-devices.

73. The system according to any of examples 56-72, wherein at least two of the implantable patient-devices are implanted at a level and coordinate operation to maintain one or more spinal corrections.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2017, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;”
U.S. Application No. 62/773,127, filed on Nov. 29, 2018, titled “SYSTEMS AND METHODS FOR ORTHOPEDIC IMPLANTS;”
U.S. Application No. 62/928,909, filed on Oct. 31, 2019, titled “SYSTEMS AND METHODS FOR DESIGNING ORTHOPEDIC IMPLANTS BASED ON TISSUE CHARACTERISTICS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titles “SYSTEMS AND METHODS FOR DESIGNING ORTHOPEDIC IMPLANTS BASED ON TISSUE CHARACTERISTICS;” and
U.S. application Ser. No. 17/100,396, filed Nov. 20, 2020, titled “PATIENT-SPECIFIC VERTEBRAL IMPLANTS WITH POSITIONING FEATURES.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A computer-implemented method for establishing and managing a patient-specific device network, the method comprising:
generating a corrective plan for multiple implantable patient-devices in a patient based on patient data of the patient; and
transmitting data according to the corrective plan to one or more of the implantable patient-devices, wherein the implantable patient-devices are configured to communicate with each other via a wireless mesh network based on the corrective plan.

2. The computer-implemented method of claim 1, wherein the implantable patient-devices autonomously operate to coordinate positioning of anatomical elements of the patient according to the corrective plan.

3. The computer-implemented method of claim 1, wherein the implantable patient-devices are configured to mechanically move anatomical elements of the patient to adjust at least one of lumbar lordosis, Cobb angles, coronal parameters, sagittal parameters, or pelvic parameters according to the corrective plan.

4. The computer-implemented method of claim 1, further comprising transmitting the corrective plan to at least one of the patient-devices that uses the corrective plan and the transmitted data to provides one or more target anatomical corrections.

5. The computer-implemented method of claim 1, further comprising transmitting the corrective plan to at least one of the patient-devices, wherein the corrective plan is configured to coordinate operation of one or more of the patient-devices.

6. The computer-implemented method of claim 1, further comprising:
selecting at least one of the patient-devices based on communication protocols, a power setting, a messaging formation, or a combination.

7. The computer-implemented method of claim 1, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

8. The computer-implemented method of claim 1, wherein at least one of the implantable patient-devices is programmed to wirelessly receive software, install and run the received software, and wirelessly transmit to software to another one of the implantable patient devices.

9. The computer-implemented method of claim 1, further comprising:
generating manufacturing data for the implantable patient devices based on the correct plan; and
manufacturing the implantable patient devices according to the manufacturing data.

10. The computer-implemented method of claim 1, wherein the multiple implantable patient-devices are programmed to reposition vertebrae of a spine of the patient to achieve one or more spinal corrections according to the corrective plan.

11. The computer-implemented method of claim 1, further comprising:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

12. The computer-implemented method of claim 1, further comprising:
identifying a newly available implantable patient-device in the patient;
authenticating the newly available implantable patient-device; and
allowing the authenticated newly available implantable patient-device to become part of the wireless mesh network.

13. The computer-implemented method of claim 1, further comprising:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

14. The computer-implemented method of claim 1, wherein the corrective plan includes target ranges of corrections values, wherein the multiple implantable patient-devices are configured to corporate to position anatomical features to keep correction values of the patient within the target ranges of corrections values.

15. The computer-implemented method of claim 1, wherein the implantable patient-devices include a first implanted device and a second implanted device, wherein the first implanted device sends wirelessly transmitted commands to the second implanted device accordingly to a spinal correction plan.

16. The computer-implemented method of claim 1, further comprising:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

17. The computer-implemented method of claim 1, wherein at least of the implantable patient-devices includes a controller programmed to autonomously establish and manage a network among the implanted patient-devices.

18. The computer-implemented method of claim 1, wherein at least two of the implantable patient-devices are implanted at a level and coordinate operation to maintain one or more spinal corrections.

19. A non-transitory computer-readable storage medium storing instructions that, when executed by a computing system, cause the computing system to perform operations for establishing and managing a patient-specific device network, the operations comprising:
generating a corrective plan for multiple implantable patient-devices in a patient based on patient data of the patient; and
transmitting data according to the corrective plan to one or more of the implantable patient-devices, wherein the implantable patient-devices are configured to communicate with each other via a wireless mesh network based on the corrective plan.

20. The non-transitory computer-readable medium of claim 19, wherein the implantable patient-devices autonomously operate to coordinate positioning of anatomical elements of the patient according to the corrective plan.

21. The non-transitory computer-readable medium of claim 19, wherein the implantable patient-devices are configured to mechanically move anatomical elements of the patient to adjust at least one of lumbar lordosis, Cobb angles, coronal parameters, sagittal parameters, or pelvic parameters according to the corrective plan.

22. The non-transitory computer-readable medium of claim 19, wherein the operations further comprise transmitting the corrective plan to at least one of the patient-devices that uses the corrective plan and the transmitted data to provides one or more target anatomical corrections.

23. The non-transitory computer-readable medium of claim 19, wherein the operations further comprise transmitting the corrective plan to at least one of the patient-devices, wherein the corrective plan is configured to coordinate operation of one or more of the patient-devices.

24. The non-transitory computer-readable medium of claim 19, wherein the operations further comprise:
selecting at least one of the patient-devices based on communication protocols, a power setting, a messaging formation, or a combination.

25. The non-transitory computer-readable medium of claim 19, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

26. The non-transitory computer-readable medium of claim 19, wherein at least one of the implantable patient-devices is programmed to wirelessly receive software, install and run the received software, and wirelessly transmit to software to another one of the implantable patient devices.

27. The non-transitory computer-readable medium of claim 19, wherein the operations further comprise:
generating manufacturing data for the implantable patient devices based on the correct plan; and
manufacturing the implantable patient devices according to the manufacturing data.

28. The non-transitory computer-readable medium of claim 19, wherein the multiple implantable patient-devices are programmed to reposition vertebrae of a spine of the patient to achieve one or more spinal corrections according to the corrective plan.

29. The non-transitory computer-readable medium of claim 19, wherein the operations further comprise:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

30. The non-transitory computer-readable medium of claim 19, wherein the operations further comprise:
identifying a newly available implantable patient-device in the patient;
authenticating the newly available implantable patient-device; and
allowing the authenticated newly available implantable patient-device to become part of the wireless mesh network.

31. The non-transitory computer-readable medium of claim 19, wherein the operations further comprise:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

32. The non-transitory computer-readable medium of claim 19, wherein the corrective plan includes target ranges of corrections values, wherein the multiple implantable patient-devices are configured to corporate to position anatomical features to keep correction values of the patient within the target ranges of corrections values.

33. The non-transitory computer-readable medium of claim 19, wherein the implantable patient-devices include a first implanted device and a second implanted device, wherein the first implanted device sends wirelessly transmitted commands to the second implanted device accordingly to a spinal correction plan.

34. The non-transitory computer-readable medium of claim 19, wherein the operations further comprise:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

35. The non-transitory computer-readable medium of claim 19, wherein at least of the implantable patient-devices includes a controller programmed to autonomously establish and manage a network among the implanted patient-devices.

36. The non-transitory computer-readable medium of claim 19, wherein at least two of the implantable patient-devices are implanted at a level and coordinate operation to maintain one or more spinal corrections.

37. A system comprising:
one or more processors; and
one or more memories storing instructions that, when executed by the one or more processors, cause the system to perform a process for establishing and managing a patient-specific device network, the process comprising:
generating a corrective plan for multiple implantable patient-devices in a patient based on patient data of the patient; and
transmitting data according to the corrective plan to one or more of the implantable patient-devices, wherein the implantable patient-devices are configured to communicate with each other via a wireless mesh network based on the corrective plan.

38. The system according to claim 37, wherein the implantable patient-devices autonomously operate to coordinate positioning of anatomical elements of the patient according to the corrective plan.

39. The system according to claim 37, wherein the implantable patient-devices are configured to mechanically move anatomical elements of the patient to adjust at least one of lumbar lordosis, Cobb angles, coronal parameters, sagittal parameters, or pelvic parameters according to the corrective plan.

40. The system according to claim 37, wherein the process further comprises transmitting the corrective plan to at least one of the patient-devices that uses the corrective plan and the transmitted data to provides one or more target anatomical corrections.

41. The system according to claim 37, wherein the process further comprises transmitting the corrective plan to at least one of the patient-devices, wherein the corrective plan is configured to coordinate operation of one or more of the patient-devices.

42. The system according to claim 37, wherein the process further comprises:
selecting at least one of the patient-devices based on communication protocols, a power setting, a messaging formation, or a combination.

43. The system according to claim 37, wherein at least one of the implantable patient-devices is programmed to
detect at least one value;
determining an anatomical adjustment based on the detected at least one value and the corrective plan; and
moving from a first configuration to a second configuration to achieve the determined anatomical adjustment.

44. The system according to claim 37, wherein at least one of the implantable patient-devices is programmed to wirelessly receive software, install and run the received software, and wirelessly transmit to software to another one of the implantable patient devices.

45. The system according to claim 37, wherein the process further comprises:
generating manufacturing data for the implantable patient devices based on the correct plan; and
manufacturing the implantable patient devices according to the manufacturing data.

46. The system according to claim 37, wherein the multiple implantable patient-devices are programmed to reposition vertebrae of a spine of the patient to achieve one or more spinal corrections according to the corrective plan.

47. The system according to claim 37, wherein the process further comprises:
a first implantable patient-device having one or more sensors configured to output signals; and
a second implantable patient-device configured to wirelessly receive data associated with the outputted signals, and the second implantable patient-device moves from a first configuration to a second configuration based on the received data when implanted.

48. The system according to claim 37, wherein the process further comprises:
identifying a newly available implantable patient-device in the patient;
authenticating the newly available implantable patient-device; and
allowing the authenticated newly available implantable patient-device to become part of the wireless mesh network.

49. The system according to claim 37, wherein the process further comprises:
obtaining sensor readings from a first one of the implanted devices;
selecting at least one of the implantable patient-device to be reconfigured based on a correlation and a correction plan for a spine of the patient; and
commanding to the selected at least one implantable patient-device to move from a first configuration to a second configuration accordingly to the corrective plan.

50. The system according to claim 37, wherein the corrective plan includes target ranges of corrections values, wherein the multiple implantable patient-devices are configured to corporate to position anatomical features to keep correction values of the patient within the target ranges of corrections values.

51. The system according to claim 37, wherein the implantable patient-devices include a first implanted device and a second implanted device, wherein the first implanted device sends wirelessly transmitted commands to the second implanted device accordingly to a spinal correction plan.

52. The system according to claim 37, wherein the process further comprises:
measuring at least one value using a sensor of one of the implantable patient-devices;
determining a spinal correction for a spine of the patient based on the measured at least one value; and
causing at least one of the implantable patient-devices to move anatomical elements to achieve the determined spinal correction.

53. The system according to claim 37, wherein at least of the implantable patient-devices includes a controller programmed to autonomously establish and manage a network among the implanted patient-devices.

54. The system according to claim 37, wherein at least two of the implantable patient-devices are implanted at a level and coordinate operation to maintain one or more spinal corrections.
